(19) United States
(12) Patent Application Publication
(10) Pub. No.: US 2025/0352675 A1
(43) Pub. Date: Nov. 20, 2025

(54) CA-IX TARGETING FLUORESCENT PROBES

(71) Applicant: BRACCO IMAGING S.P.A., Milan (IT)

(72) Inventors: Roberta NAPOLITANO, Albiano d'Ivrea (IT); Francesco BLASI, Colleretto Giacosa (IT); Federica CHIANALE, Chivasso (IT); Erika REITANO, Torino (IT); Alessia ADAMO, Banchette (TO) (IT)

(73) Assignee: BRACCO IMAGING S.P.A., Milan (IT)

(21) Appl. No.: 18/872,161

(22) PCT Filed: Jul. 26, 2023
(86) PCT No.: PCT/EP2023/070676

§ 371 Date: Dec. 5, 2024

(30) Foreign Application Priority Data
Jul. 28, 2022 (EP) 22187425.8

(51) Int. Cl. A61K 49/00; C09B 23/01; C09B 23/08

(52) U.S. Cl. CPC A61K 49/0052; A61K 49/0032; C09B 23/0066; C09B 23/086

(57) ABSTRACT

The present invention relates to the field of optical imaging. More particularly, it relates to fluorescent probes targeting carbonic anhydrase nine (CA-IX) comprising near-infrared (NIR) dyes of the cyanine. The invention also relates to the methods for preparing these compounds, to pharmaceutical compositions and kits incorporating them and to methods of use them as optical diagnostic agents in imaging or therapy of diseases such as solid tumors with hypoxic tissues involving cells expressing CA-IX. The compounds have formula (I).

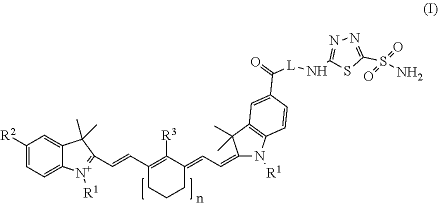

### FIELD OF THE INVENTION

The present invention relates to the field of optical imaging. More particularly, it relates to fluorescent probes able to efficiently target Carbonic Anhydrase IX enzyme (CA-IX) and comprising heptamethine cyanine dyes with near-infrared (NIR) emission. The invention also relates to methods for preparing these compounds, to pharmaceutical compositions and kits incorporating them and to methods of use them as optical diagnostic agents in imaging or therapy of diseases, particularly solid tumors with hypoxic tissues involving cells expressing CA-IX.

### BACKGROUND ART

Dyes are chemical entities that absorb photons of a specific wavelength upon light excitation and re-emit some of that energy, depending on quantum efficiency, usually at a longer wavelength. Particularly, cyanine dyes are fluorescent organic molecules characterized by a delocalized electron system that spans over a polymethine bridge and is confined between two nitrogen atoms. Given the favorable optical properties, low toxicity, and good solubility in aqueous media, cyanine dyes can be used as contrast agents for biomedical imaging. Cyanine dyes emitting in the Near-Infra Red (NIR) region (700-900 nm) are particularly useful for biomedical imaging applications due to the high penetration depth compared to dyes with fluorescence emission in the visible spectrum.

Among the NIR dyes currently used for biomedical imaging, Indocyanine green (ICG) is a medicinal product approved for human use that may accumulate in pathological regions thanks to different homing mechanisms. Fluorophores like Fluorescein and ICG distribute in tumor tissues by a combination of passive diffusion and enhanced permeability and retention (EPR) effect (Onda N. et al., Int J Cancer 2016, 139, 673-682). However, this first generation of fluorescent contrast agents requires a large mass dose (>1 mg/kg) for proper visualization. Moreover, false positive and false negative responses are common clinical findings associated with the use of these contrast agents (Tummers Q. et al., PlosOne 2015).

A second generation of contrast agents for optical imaging is currently under development, exploiting the use of a dye conjugated to a targeting agent to improve sensitivity and specificity of detection. Molecular vectors such as small molecules, peptides, mAbs, antibody fragments, confer tumor selective properties to the probes, whereas the fluorescent dyes work as detection moieties for spatial localization (Gioux S. et al., Mol Imaging 2010, 9(5), 237-255). However, the in vivo behavior of dye-biomolecule conjugates may be strongly affected by the biological properties of the fluorescent moiety. For example, small structural modifications of cyanine Cy5 strongly modulate the accumulation in tumor and off-target tissues of the relative bioconjugates (Bunschoten A. et al., Bioconjugate Chem. 2016, 27, 1253-1258). Fluorescent contrast agents with low non-specific accumulation and high selectivity for the target tissue would be preferable for applications in living organisms.

Several examples of known cyanine dyes conjugated with low molecular weight targeting moieties are disclosed in the background art. For instance, one of the targets of utmost interest for its potential for clinical settings is Carbonic Anhydrase IX (CA-IX).

CA-IX is a homo-dimeric glycoprotein located on the cell surface (Hilvo et al., J Biol Chem 2008; 283(41): 27799-27809) and is a member of the large carbonic anhydrases (CAs) family of zinc metalloenzymes. These enzymes are involved in the maintenance of cellular acid-base homeostasis through the catalysis of the reversible hydration of carbonic dioxide (CO₂+H₂O), which is converted to protons and bicarbonate (HCO³⁻+H⁺), leading to a local decrease in pH.

To date, sixteen CA isoforms have been characterized in mammals, which differ in their cellular localization, catalytic activity, susceptibility to different inhibitors and tissue-specific distribution.

Among them, CA-IX has been demonstrated to be an excellent biomarker of the hypoxic response in tumors, because its gene expression is promoted by the master regulator of hypoxia, named Hypoxia Inducible Factor 1 (HIF-1), believed to be involved in maintaining the acidic environment of hypoxic cells (Wykoff et al., Cancer Res. 2000, 60, 7075-7083). Tumor hypoxia, mostly resulting from poor perfusion and anemia, is one of the key factors in inducing the development of cell clones with an aggressive and treatment-resistant phenotype that leads to rapid progression and poor prognosis in several cancer types. Cancer cells, in fact, survive in a hostile environment changing their gene expression, and among them especially genes involved in pH control.

Indeed, CA-IX plays an important role in the growth and metastasis of numerous tumors (including renal cancer, cervical cancer, colon cancer, prostate cancer, breast cancer, head and neck tumors, etc), because its catalytic activity contributes to the reduction of the extracellular pH, producing an acid microenvironment which increases cancer cell proliferation and invasion.

Unlike other CAs, many studies have demonstrated that CA-IX is expressed only in few normal tissues (namely the intestinal and stomach mucosa, gallbladder and testis), whereas it becomes overexpressed in many types of cancer cells, especially in solid tumors, characterized by insufficient oxygen supply. CA-IX thus represents an established biomarker of tumor hypoxia which plays a pivotal role in carcinogenesis and it is considered as a hallmark of the disease (Muz et al., Hypoxia (Auckl) 2015; 3:83-92).

CA-IX targeting using specific tools could open new important fields to improve the conventional therapies and the early diagnosis and prognosis of malignant tumors.

The classes of CA-IX-targeting agents developed so far, for both imaging and/or therapeutic applications, include monoclonal antibodies (e.g., G250, M75) or mini-antibodies (e.g., A3 and CC7) and small chemical compounds, such as inorganic ions, sulfonamide-based compounds, phenols and coumarins. Some agents belonging to these classes of compounds are currently under clinical development.

In particular, the monoclonal antibodies (mab) M75 and G250 represent the first solutions developed to target CA-IX enzyme. Mab M75 binds to CA-IX's PG-like domain on the N-terminus of the target, whereas mab G250 interacts with CA-IX's catalytic domain. A chimeric version of G250 (designated cG250), labelled with the radionuclide ¹²⁴I, was developed for the detection of clear cell Renal Cell Carcinoma (ccRCC). The use of the antibody cG250 in a method of diagnosing, predicting and/or classifying a cancer disease including quantification of CA-IX expression is for instance described in WO2014/128258.

Although, typically, monoclonal antibodies have been considered as the ligands of choice for most tumor targeting applications, it is becoming increasingly clear that they are subjected to many disadvantages. In fact, antibodies are characterized by slow and inefficient tumor penetration and long blood residence, which requires the use of long-lived radioisotopes and imaging at late time points, exposing patients to a high radiation burden. Indeed, ¹²⁴I-cG250 reaches tumor/blood ratios suitable for imaging only 2-7 days after injection into the patient. Additionally, monoclonal antibodies may be immunogenic, precluding repeated administration for routine diagnostic procedures.

These problems could be circumvented with the use of small molecules. Unlike large macromolecules, small molecules clear rapidly from circulation and thus reach tumor/blood ratios suitable for imaging at early time-points. This fact in turn allows physicians to obtain diagnostic information much more quickly than with antibody-based imaging agents.

Among small chemical compounds targeting Carbonic Anhydrase, the best investigated and most robust class of inhibitors are the sulfonamides (i.e. acetazolamide) due to their high affinity, availability and ease of chemical manipulation.

While they have been in clinical use for decades for conditions such as glaucoma, seizures, altitude sickness and as a diuretic, only recently they have also been considered as antitumor agents for their capability to inhibit CA-IX. The first compound to arrive in Phase I and II clinical trials, alone or in combination with other antitumor agents, was Indisulam, but it has been discontinued. The only CA inhibitor (CAI) currently in clinical development is SLC-0111, an ureidobenzenesulfonamide that has successfully completed a phase I study, to determine safety and tolerability in patients with advanced solid tumors (McDonald et al., Am J Clin Oncol 2020; 43(7): 484-490).

For imaging purposes, CAIs have been labeled with various positron emitting isotopes (e.g., ¹⁸F, ⁶⁴Cu, ⁶⁸Ga, ¹¹¹In) or with ^{99m}Tc in order to obtain PET/SPECT agents.

Acetazolamide is an FDA-approved drug used to treat glaucoma, epilepsy, altitude sickness, periodic paralysis, heart failure, etc. Acetazolamide is a first-generation carbonic anhydrase inhibitor, hence causing the accumulation of carbonic acid, and it is known to strongly bind the Zn ion present in the catalytic pocket thanks to its terminal sulfonamide group, deprotonated as —SONH⁻ at physiological pH.

It is possible to hydrolyze the acetyl group of acetazolamide and replace it with different linkers, for example by acylation of the amino group, still maintaining the inhibition activity and a medium/strong binding to the enzyme, depending of course on the type of substituent.

In 2015, Wichert et al. (Nat Chem 2015; 7(3): 241-249) described the selection of novel low-nanomolar binders for CA-IX by an innovative chemical library technology for affinity maturation of ligands against a defined target. Among them, the acetazolamide derivative named “4a ligand” was identified by modification via acyl chloride and click chemistry of acetazolamide, after hydrolysis of the acetyl moiety in strong acidic aqueous media. Ligand 4a displayed high affinity for CA-IX (16.7 nM, measured by SPR) and its use as delivery vehicle for tumor targeting has been reported.

Acetazolamide and other sulfonamides have been also conjugated to various NIR fluorescent dyes with the aim of imaging hypoxia-induced CA-IX expression in tumor cells. Indeed, among the fluorescent dyes targeting CA-IX reported in literature functionalized with different sulfonamides, even cyclic secondary amide as saccharine, the best results in terms of binding have always been obtained by derivatization of acetazolamide. A pilot in vivo experiment illustrated the potential of NIR fluorescent CAIs and fluorescence molecular tomography (FMT) imaging for non-invasive quantification of CA-IX expression (Groves et al., Bioorg Med Chem Lett 2012; 22:653-657).

The preferred sites of conjugation to the cyanine dyes disclosed in the art are represented by the positions 1 or 3 of the indolenine groups for linear Cy5-Cy7 cyanines and Cy7 cyanines with cyclohexenyl central ring, typically by conjugation at an alkyl chain functionalized with a carboxylic acid or ester. Cy7 cyanines with cyclohexenyl central ring can be also conjugated to the acetazolamide targeting moiety at the central (meso) position of the heptamethine scaffold, typically by attachment to a phenyl ring, optionally attached to the heptamethine chain through an interposed oxygen atom and functionalized in para with a carboxy, carboxamido or ester group, optionally by interposition of a linker. These compounds generally have a symmetrical structure, bearing —SO₃H or alkyl-SO₃H groups in the other positions.

One example of such derivatives is disclosed in WO2017/161195 (On Target Laboratories) which describes the compound Az-8AOA-LMNIR2, also known as HypoxyFluor-1, currently under development as fluorescent probe for targeted imaging of tumors having high expression of Carbonic Anhydrase (CA) IX enzyme, like optical imaging and surgery involving CA-IX positive tissues and tumors. This compound is also mentioned for instance in Mahalingam S. M. et al., Bioconjugate Chem 2018, 29, 3320-3331, where its use for fluorescence imaging of hypoxic tumors is described in detail.

The conjugation at the central position, however, in several cases, suffers from higher lability of the central substituent, that could be removed in vivo by reaction with endogenous nucleophiles (i.e., it is susceptible of in vivo nucleophilic substitution), causing the loss of the targeting unit and leading to a potential in vivo degradation and de-activation of the probe. This drawback has been partially resolved by replacing the central C—O bond with a C—C bond, much more stable in vivo, but in this case the water solubility of the dye tends to dramatically decrease due to a higher tendency of aggregation.

In general, the use of tumor-targeted fluorescence dyes to help surgeons identify otherwise undetected tumor nodules, decrease the incidence of cancer-positive margins, and facilitate localization of malignant lymph nodes, has been demonstrated considerably promising for improving cancer debulking surgery. Unfortunately, the repertoire of available fluorescent dyes targeting CA-IX expressing tumors is not yet extensive and diversified to permit identification of all types of hypoxic tumoral cells, raising the need to develop additional CA-IX-specific fluorescent dyes that, taking advantage from specific targeting at the biomolecular level, may ensure visualization of all malignant lesions and precise tumor excision during cancer surgeries.

Thus, despite the efforts made so far, there is still the urgent and unmet need to develop efficient fluorescent probes targeting CA-IX with high affinity and specificity in order to be used in diagnosis and/or therapy.

Differently from the above-mentioned probes, the compounds of the present invention are characterized by Cy7 cyanines conjugated to an acetazolamide moiety at an alternative position of the cyanine scaffold, namely at position 5 of at least one indolenine ring. The probes with conjugation in said position have surprisingly shown a higher affinity for the target CA-IX enzyme, besides a higher stability during their preparation.

### SUMMARY OF THE INVENTION

Generally, object of the present invention is to provide CA-IX targeting fluorescent probes useful as contrast agents for optical imaging and aimed at solving the above-mentioned issues. Particularly, the present invention provides fluorescent probes with optimal properties for different molecular imaging applications, being able to interact with CA-IX expressing cells, to accumulate in pathological cells and tissues and to specifically display a fluorescent signal in correspondence to pathological tissues with high signal-to-noise ratio and improved imaging efficacy at low mass doses.

The fluorescent probes of the invention are characterized by a heptamethine cyanine dye conjugated to an acetazolamide moiety at a specific position of the cyanine scaffold, namely at position 5 of at least one of the indolenine rings, affording strong binding results to the target and unexpectedly displaying an optimal in vitro and in vivo behaviour, thus being particularly suitable for molecular imaging.

In detail, among the several advantages that can be achieved by means of the present compounds, the following features can be highlighted for instance: high selectivity for the target tissue and low accumulation due to non-specific interaction with other tissues, high solubility in water, low binding to albumin, negligible or no observed adverse reactions after systemic administration, good chemical and optical stability in plasma after administration. A further aspect of the invention relates to such fluorescent probes for use as diagnostic agents, in particular for use in optical imaging of a human or animal organ or tissue, for use in a method of optical imaging, wherein the imaging is a tomographic imaging of organs, monitoring of organ functions including angiography, urinary tract imaging, bile duct imaging, nerve imaging, intraoperative cancer identification, fluorescence-guided surgery, fluorescence life-time imaging, short-wave infrared imaging, fluorescence endoscopy, fluorescence laparoscopy, robotic surgery, open field surgery, laser guided surgery, or a photoacoustic or sonofluorescence method.
Moreover, the invention relates to a manufacturing process for the preparation of the provided compounds and/or pharmaceutically acceptable salts thereof, and to their use in the preparation of a diagnostic agent.
According to a further aspect, the invention relates to a pharmaceutically acceptable composition comprising at least one compound of the invention, or a pharmaceutically acceptable salt thereof, in a mixture with one or more physiologically acceptable carriers or excipients. Said compositions are useful in particular as optical imaging agents to provide useful imaging of human or animal organs or tissues.
In another aspect, the present invention refers to a method for the optical imaging of a body organ, tissue or region by use of an optical imaging technique that comprises the use of an effective dose of a compound of the invention.

### DESCRIPTION OF THE INVENTION

Accordingly, a first aspect of the invention relates to compound of formula (I), or a pharmaceutically acceptable salt thereof,

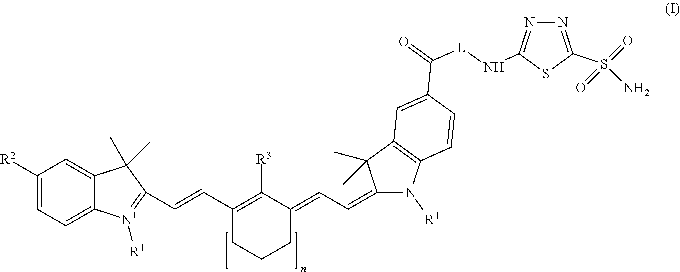

wherein
each R¹ is independently a straight or branched C₁-C₁₀ alkyl substituted by a group —SO₃H;
R² is selected from —SO₃H, a group —CONH—Y wherein Y is a straight or branched C₁-C₁₀ alkyl substituted with at least two hydroxyl groups, and a group of formula (II)

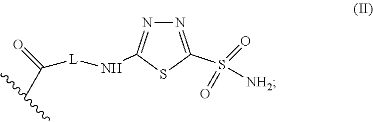

R³ is selected from hydrogen and a group phenyl or —O-phenyl optionally substituted with —SO₃H;
L is a bond or a linker;
n is an integer equal to 0 or 1.

Preferably the linker L is a group —NH—(CH₂)ₚ—CO— or a diradical of one or more moieties selected from the group consisting of an amino acid, such as for instance glycine, alanine, β-alanine, lysine, homolysine, ornithine, glutamic acid, aspartic acid and the like; a peptide comprising from 2 to 10 amino acids in L or D configuration; 4-aminomethylbenzoic acid; cysteic acid; a polyethylene glycol, such as a group of formula —NH—(O—CH₂—CH₂)ₚ- or —NH—(O—CH₂—CH₂)ₚ—CO— or derivatives thereof; amino-polyethylene glycol-carboxylic acid; diaminobutyric acid; and diaminopropionic acid; or it is a group -L₁-L₂- wherein L₁ is a diradical of a diamine, such as for instance an amino-polyethylene glycol amine of formula —NH—(O—CH₂—CH₂)ₚ—NH— or a diradical of ethylenediamine, propylenediamine, putrescine, spermidine, spermine, hexanediamine and the like; and L₂ is a diradical of a dicarboxylic acid, such as for instance succinic acid, glutaric acid, suberic acid, adipic acid and the like; wherein p is an integer comprised between 1 and 20.

More preferably, L is selected from a group of formula —NH—(CH₂)ₚ—CO—; a polyethylene glycol of formula —NH—(O—CH₂—CH₂)ₚ—CO—; and a diradical comprising from one to five amino acids, wherein p is an integer comprised between 1 and 20.

The present invention also relates to methods for preparing the compounds of formula (I) by means of synthetic transformations steps.

The invention further comprises compounds of formula (I) for use as fluorescent agents for the detection of a tumor margin in guided surgery.

### DESCRIPTION OF THE FIGURES

FIG. 1 shows the binding curves of the representative compounds 1, 2 and 3 of the invention and the reference compound HF-1 to CA-IX expressing HT-29 cells.

FIG. 2 shows the competition exerted by the anti CA-IX antibody clone MSC8 against the binding to HT-29 cells of representative compounds 1, 2, and 3. In these experiments, compounds 1, 2, and 3 were used at 0.1 μM concentrations, alone or in combination with MSC8 at either 0.01, or 0.1, or 1 μM.

FIG. 3 shows the time-dependent uptake of representative compounds 1, 2 and 3 and corresponding unconjugated dyes to HT-29 cells, at 37° C. and after incubation on ice.

FIG. 4 shows a linear representation of cellular uptake of representative compounds 1, 2 and 3 at 37° C.

FIG. 5 shows the in vivo tumor-to-background ratio (TBR) of representative compounds 1, 2 and 3 and of the reference compound HF-1 from images acquired 24 hours after administration.

FIG. 6 shows (a) the ex vivo tumor-to-background ratio (TBR) and (b) the organs-to-muscle ratio of representative compounds 1, 2 and 3 and of the reference compound HF-1 from samples collected 24 hours after administration.

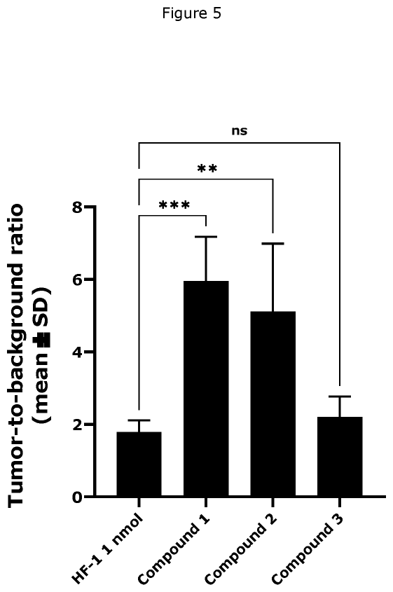

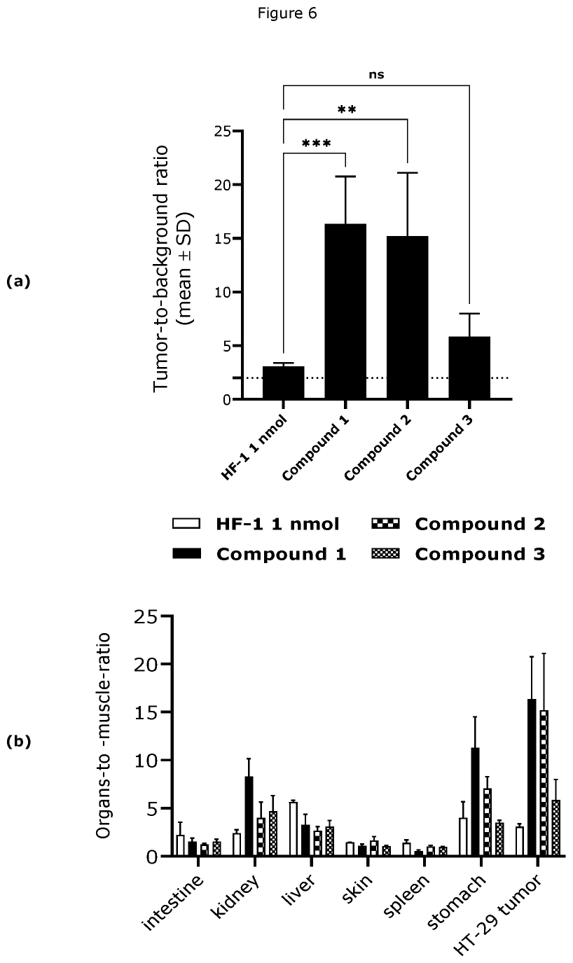

TBR statistical analysis in FIGS. 5 and 6(a) was performed using 1-way-ANOVA, Dunnett's multiple comparison test, where p<0.05 indicated significant values different from reference HF-1 (1 nmol/mouse).

### Definitions

In the present description, and unless otherwise provided, the following terms and phrases as used herein are intended to have the following meanings.

The term “diradical” refers to a chemical group wherein the hydrogen atoms at two terminal portions of the molecule are removed to form a bond.

The expression “straight or branched C₁-C₁₀ alkyl” refers to an aliphatic hydrocarbon radical group, which may be a straight or branched chain, having from 1 to 10 carbon atoms in the chain. For instance, “C₁-C₈ alkyl” comprises within its meaning a linear or branched chain comprising from 1 to 8 carbon atoms. Representative and preferred alkyl groups include methyl, ethyl, n-propyl, iso-propyl, n-butyl, tert-butyl, pentyl, hexyl and the like. Unless otherwise specified, the straight or branched alkyl is a monovalent radical group. In some cases it may be a “bivalent” or “multivalent” radical group, wherein two or more hydrogen atoms are removed from the above hydrocarbon radical group and substituted, e.g. methylene, ethylene, iso-propylene groups and the like. In such cases, the expression “straight or branched C₁-C₁₀ alkylene” is used.

The term “hydroxyalkyl” refers to any of the corresponding alkyl chain wherein one or more hydrogen atoms are replaced by hydroxyl groups.

The term “protecting group” (Pg) designates a protective group adapted for preserving the function of the group to which it is bound. Specifically, protective groups are used to preserve amino, hydroxyl or carboxyl functions. Appropriate protective groups may include, for example, benzyl, carbonyl, such as formyl, 9-fluoromethyloxycarbonyl (Fmoc), benzyloxycarbonyl (Cbz), t-butoxycarbonyl (Boc), isopropyloxycarbonyl or allyloxycarbonyl (Alloc), alkyl, e.g. tert-butyl or triphenylmethyl, sulfonyl, acetyl groups, such as trifluoroacetyl, benzyl esters, allyl, or other substituents commonly used for protection of such functions, which are well known to the person skilled in the art (see, for instance, the general reference T. W. Green and P. G. M. Wuts, Protective Groups in Organic Synthesis, Wiley, N.Y. 2007, 4^{th} Ed., Ch. 5).

Moreover, the invention comprises also the precursors or intermediates compounds suitable for the preparation of a desired compound of formula (I) or salts thereof. In such intermediates any functional group, such as a carboxylic acid or carboxamide, can be protected with an appropriate protecting group (Pg) as defined above, preferably with alkyl or ester groups. If necessary, also hydroxyl groups of Y groups can be protected with an appropriate protecting group (Pg) during the preparation of the compounds of formula (I), forming for instance acetoxy, alkoxy or ester groups.

The expression “coupling reagent” refers to a reagent used for instance in the formation of an amide bond between a carboxyl moiety and an amino moiety. The reaction may consist of two consecutive steps: activation of the carboxyl moiety and then acylation of the amino group with the activated carboxylic acid. Non limiting examples of such coupling agents are selected from the group consisting of: carbodiimides, such as N,N′-diisopropylcarbodiimide (DIC), N,N′-dicyclohexylcarbodiimide (DCC), 1-ethyl-3-(3-dimethylaminopropyl) carbodiimide (EDAC), 1-ethyl-3-(3-dimethylaminopropyl)carbodiimide (EDC) and 1-ethyl-3-(3-dimethylaminopropyl)carbodiimide (WSC); phosphonium reagents, such as (benzotriazol-1-yloxy)tris(dimethylamino)phosphonium hexafluorophosphate (BOP), (benzotriazol-1-yloxy)tripyrrolidinophosphonium hexafluorophosphate (PyBOP), 7-azabenzotriazol-1-yloxy-tripyrrolidino-phosphonium hexafluorophosphate (PyAOP), [ethyl cyano(hydroxyimino)acetato-02]tri-1-pyrrolidinylphosphonium hexafluorophosphate (PyOxim), bromotripyrrolidinophosphonium hexafluorophosphate (PyBrOP) and 3-(diethoxyphosphoryloxy)-1,2,3-benzotriazin-4(3H)-one (DEPBT); and aminium/uronium-imonium reagents, such as N,N,N′,N′-tetramethyl-O-(benzotriazol-1-yl)uronium tetrafluoroborate (TBTU), N,N,N′,N′-tetramethyl-O-(1H-benzotriazol-1-yl)uronium hexafluorophosphate (HBTU), N,N,N′,N′-tetramethyl-O-(7-azabenzotriazol-1-yl)uronium hexafluorophosphate (HATU), 0-(1H-6-chlorobenzotriazole-1-yl)-1,1,3,3-tetramethyluronium hexafluorophosphate (HCTU), 1-[1-(cyano-2-ethoxy-2-oxoethylidene-aminooxy)-dimethylamino-morpholino]-uronium hexafluorophosphate (COMU), 2-(2,5-dioxopyrrolidin-1-yl)-1,1,3,3-tetramethylisouronium tetrafluoroborate (TSTU), N,N,N′,N′-tetramethyl-O—(N-succinimidyl)uronium hexafluorophosphate (HSTU) and fluoro-N,N,N′,N′-tetramethylformamidinium hexafluorophosphate (TFFH) or other compounds well known to the person skilled in the art.

The expression “activated carboxylic acid” refers to a derivative of a carboxyl group that is more susceptible to nucleophilic attack than a free carboxyl group; suitable derivatives may include for instance acid anhydrides, thioesters, acyl halides, NHS ester and sulfo NHS esters.

The terms “moiety” or “residue” are herewith intended to define the residual portion of a given molecule once properly attached or conjugated, either directly or through a suitable linker, to the rest of the molecule.

The term “imaging agent” refers to a detectable entity that can be used in in vitro, ex vivo or in vivo visualization or detection of a biological element including cells, biological fluids and biological tissues originating from a live mammal patient, and preferably, human patient, as well as human body organ, regions or tissues, when the said detectable entity is used in association with a suitable diagnostic imaging technique.

Preferably, the fluorescent probes of the invention are able to selectively link tumor cells or tissues expressing CA-IX. In particular they are able to link tumors selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer. In addition, the fluorescent probes of the invention are able to link metastatic spreads of the above-mentioned cancers in tissues and organs different from the primary source.

Furthermore, the fluorescent probes of the invention are able to link pre-neoplastic lesions and dysplasia in different tissues and organs.

### Linker (L)

According to the invention, L is a linker, optionally present, that separates the CA-IX targeting ligand (acetazolamide) from the dye.

The presence of a linker is particularly useful for some embodiments where the acetazolamide moiety and the dye risk adversely interacting with each other. Moreover, the presence of the linker may be advantageous when the dye is relatively large and may interfere with the binding of the targeting moiety to the target site.

The linker can be either flexible (e.g., including linear alkyl chains) or rigid (e.g., including amino acids with aryl groups) so that the dye is oriented away from the target. The linker can also modify pharmacokinetic and metabolism of the conjugates of formula (I) used as imaging agents in a living organism.

Hydrophilic linkers may reduce the interaction with plasma proteins, reduce blood circulation time and facilitate excretion. For example, if the linker is a polyethyleneglycol (PEG) moiety, the pharmacokinetics and blood clearance rates of the imaging agent in vivo may be altered. In such embodiments, the linker can improve the clearance of the imaging agent from background tissue (i.e., muscle, blood) thus giving a better diagnostic image due to high target-to-background contrast. Moreover, the introduction of a particular hydrophilic linker may shift the elimination of the contrast agent from hepatic to renal, thus reducing overall body retention.

Therefore, in one preferred embodiment, the linker, when present, is a group —NH—(CH₂)ₚ—CO— or a diradical of one or more moieties selected from the group consisting of an amino acid, such as for instance glycine, alanine, β-alanine, lysine, homolysine, ornithine, glutamic acid, aspartic acid and the like; a peptide comprising from 2 to 10 amino acids in L or D configuration; 4-aminomethylbenzoic acid; cysteic acid; a polyethylene glycol such as a group of formula —NH—(O—CH₂—CH₂)ₚ— or —NH—(O—CH₂—CH₂)ₚ—CO— or derivatives thereof; amino-polyethylene glycol-carboxylic acid; diaminobutyric acid; and diaminopropionic acid; or it is a group -L₁-L₂- wherein L₁ is a diradical of a diamine, such as for instance amino-polyethylene glycol amine of formula —NH—(O—CH₂—CH₂)ₚ—NH— or a diradical of ethylenediamine, propylenediamine, putrescine, spermidine, spermine, hexanediamine and the like; and L₂ is a diradical of a dicarboxylic acid, such as for instance succinic acid, glutaric acid, suberic acid, adipic acid and the like; wherein p is an integer comprised between 1 and 20.

More preferably, L is selected from a group —NH—(CH₂)ₚ—CO—; a polyethylene glycol of formula —NH—(O—CH₂—CH₂)ₚ—CO—; and a diradical of from one to five amino acids, wherein p is an integer comprised between 1 and 20.

The compounds of the above formula (I) may have one or more asymmetric carbon atoms, otherwise referred to as chiral carbon atoms, and may thus give rise to diastereomers and optical isomers. Unless otherwise provided, the present invention further includes all such possible diastereomers as well as their racemic mixtures, their substantially pure resolved enantiomers, all possible geometric isomers, and pharmaceutically acceptable salts thereof.

The present invention further relates to compounds of the above formula (I) in which the sulfonyl groups of R¹, R² and/or R³ may be in the form of a negatively charged ion or a pharmaceutically acceptable salt.

### DETAILED DESCRIPTION OF THE EMBODIMENTS

In a preferred embodiment the invention relates to a compound of formula (I) wherein R² is a group —SO₃H.

In another preferred embodiment the invention relates to a compound of formula (I) wherein R² is a group —CONH—Y, wherein Y is selected from the group consisting of

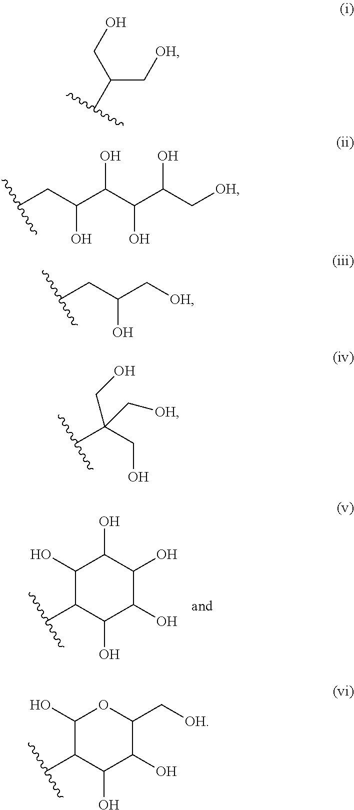

More preferably, the invention relates to a compound of formula (I) wherein R² is a group —CONH—Y and Y is a group of formula (ii) as defined above. Preferably, the group (ii) has the following stereochemical configuration, obtained by using a D-glucamine in the preparation of the compounds:

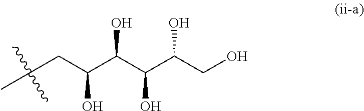 [SMILES: *C[C@H](O)[C@@H](O)[C@H](O)[C@H](O)CO]

In another preferred embodiment the invention relates to a compound of formula (I) wherein R² is a group (II), as defined above. More preferably, R² is a group of the following formula (IIa):

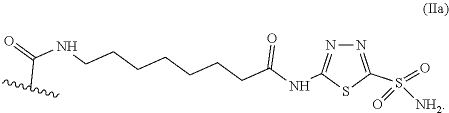 [SMILES: *C(=O)NCCCCCCCC(=O)Nc1nnc(S(N)(=O)=O)s1]

In another preferred embodiment both R¹ are a group —(CH₂)₄—SO₃H.

In another preferred embodiment, L is a group —NH—(CH₂)₇—CO—, as represented by the following formula (Ia)

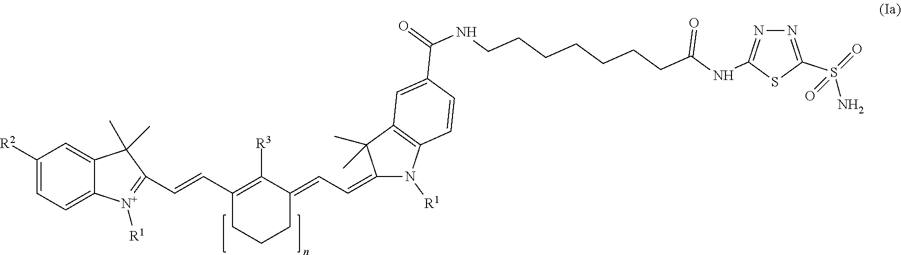

wherein R¹, R², R³ and n are as defined above.

In one preferred embodiment n is 0 and R³ is hydrogen, as represented by the following formula (Ib)

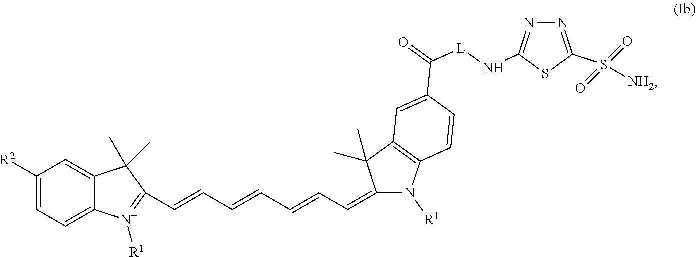

wherein R¹, R² and L are as defined above.

In another preferred embodiment n is 1 and R³ is a group —O-phenyl optionally substituted with —SO₃H, as represented by the following formula (Ic)

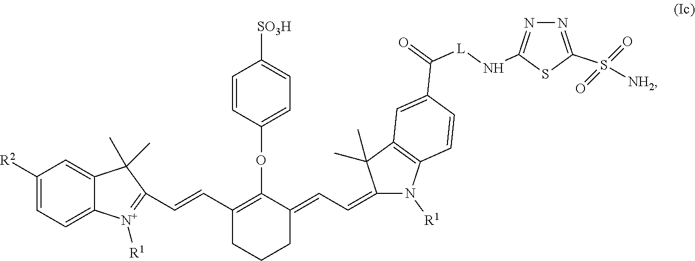

wherein R¹, R² and L are as defined above.

Especially preferred are the compounds of formula (I) listed in Table I.

Table I also reports the structure of the reference compound Hypoxyfluor-1 (HF-1) a known fluorescent probe with affinity for CA-IX.

TABLE I
Preferred compounds of formula (I) and reference compounds HF-1

| | |
|---|---|
| HF-1 (Reference) | 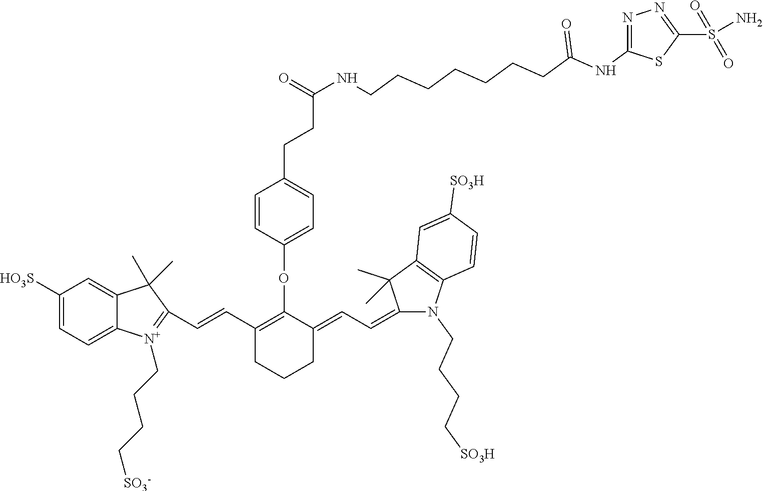 |
| Compound 1 | 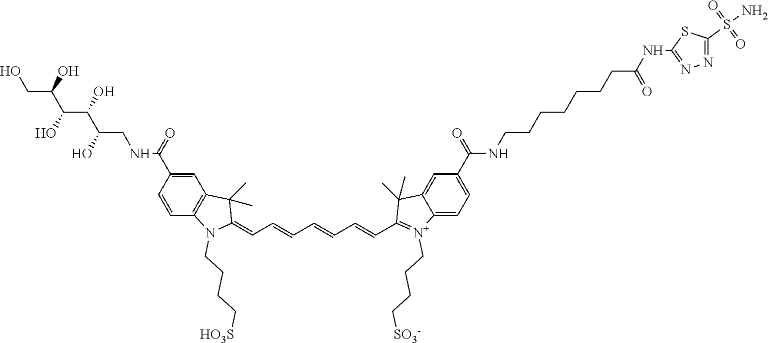 |
| Compound 2 | 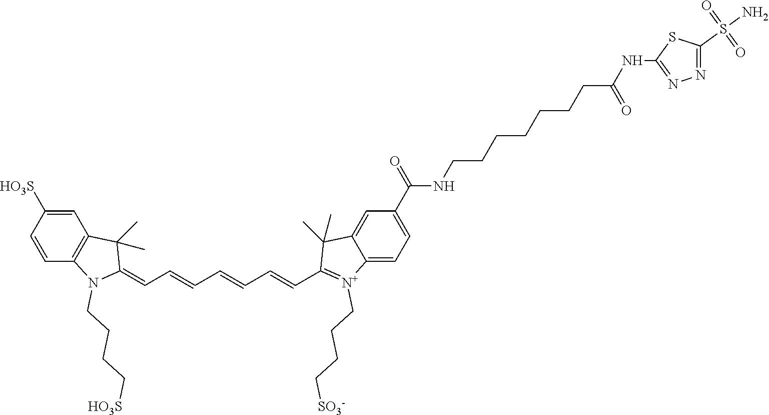 |
| Compound 3 | 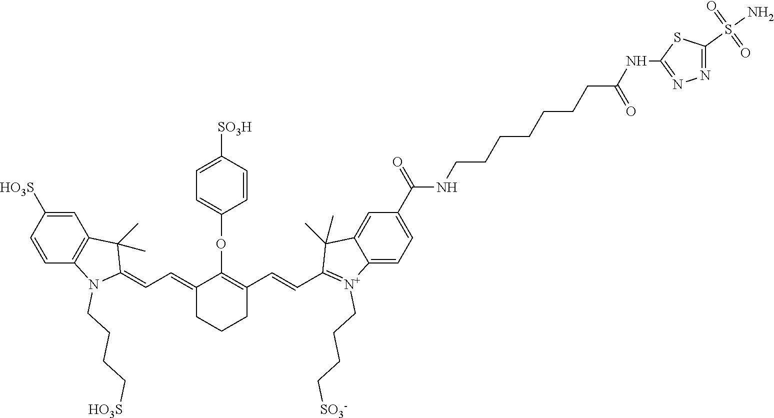 |

The present invention is also directed to methods for synthesizing the compounds of formula (I) prepared as illustrated in the following description. The compounds of the invention are useful as imaging agents in the detection of tumors in both humans and animals. Accordingly, the invention provides the compounds of formula (I) as defined above for use as fluorescent probes for the detection and demarcation of a tumor tissue during diagnostic, interventional imaging and intraoperative procedures, in particular wherein said tumor is a tumor showing an increased or variable expression of CA-IX. Preferably the imaged subject is a human.

The invention also provides a compound of formula (I) for use as fluorescent probe as defined above, wherein the detection and demarcation of the tumor tissue is carried out under NIR radiation.

Preferably, the fluorescent probes of the invention are able to selectively link cells or tissues of tumors expressing CA-IX selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer, including both primary tumors and regional and distant metastases.

The probes of the invention are able to identify in vivo a diseased tissue in a subject in need thereof. This can be accomplished by administering a compound of formula (I) as defined above and irradiating in vivo a body of the subject in need containing the diseased tissue with light having at least one excitation wavelength in the NIR range from about 650 nm to about 850 nm. Fluorescence emitted from said administered compound which is specifically bound to the diseased tissue in the body part in response to the at least one excitation wavelength is directly viewed to determine location and/or surface area of the diseased tissue in the subject.

In detail, the present invention also provides the compounds of formula (I) for use in a method for detecting the possible presence of a disease in a subject comprising the steps of:
administering to a subject in need of diagnosis an amount of a compound of formula (I) as defined above for a time and under conditions that allow for binding said compound to CA-IX expressing cells;
measuring a signal from said compound in a biological sample;
comparing said signal with at least one control data set comprising signals from the compound of formula (I) contacted with a biological sample that does not comprise the target cell type, for indicating the possible presence of a disease.

The present invention further provides the compounds of formula (I) as imaging agents for use in a method of imaging tissues and cells comprising the steps of:
contacting the tissues or cells with a compound of formula (I) as defined above;
irradiating the tissues or cells at a wavelength absorbed by the imaging agent;
detecting a near-infrared emission using a fluorescent camera.

In particular, in a preferred embodiment the invention provides a compound of formula (I) for use in a method of performing imaging guided surgery on a subject comprising the steps of:
administering a composition comprising a compound of formula (I) as defined above under conditions and for a time sufficient for the compound to accumulate at a given surgical site;
illuminating and visualizing said compound using near-infrared light;
performing surgical resection of the areas that fluorescence upon excitation by the near-infrared light.

A further aspect of this invention relates to a pharmaceutical composition comprising a fluorescent probe of formula (I) as defined above, or a salt thereof, and one or more pharmaceutically acceptable adjuvants, excipients or diluents.

Another aspect of this invention relates to a diagnostic kit comprising a compound of formula (I) as defined above. In addition, the kit can contain additional adjuvants for implementing the biomedical optical imaging application. These adjuvants are, for example, suitable buffers, vessels, detection reagents or directions for use. The kit preferably contains all materials for an intravenous administration of the compounds of the invention.

An effective amount of a compound of the invention may be administered by different routes prior to the imaging procedure, based on the disease to be treated and the location of the suspected disease to be diagnosed.

For instance, it can be administered to the organ or tissue to be imaged by a topical route, e.g. transdermally, an enteral route, e.g. orally, or a parenteral route, e.g. intradermally, subcutaneously, intramuscularly, intraperitoneally or intravenously.

In some embodiments the compounds of the invention can be administered by topically spraying or nebulizing pharmaceutical compositions comprising them and/or specifically formulated for that use.

The compositions are administered in doses effective to achieve the desired optical image of a tumor, tissue or organ, which can vary widely, depending on the compound used, the tissue subjected to the imaging procedure, the imaging equipment being used and the like.

The exact concentration of the imaging agents in a pharmaceutical composition is dependent upon the experimental conditions and the desired results, but typically may range between 1 μM to 0.1 mM. The optimal concentration is determined by systematic variation until satisfactory results with minimal background fluorescence are obtained. Once administered, the imaging agents of the invention are exposed to a light, or other form of energy, which can pass through a tissue layer. Preferably the radiation wavelength or waveband matches the excitation wavelength or waveband of the photosensitizing agent and has low absorption by the non-target cells and the rest of the subject, including blood proteins. Typically, the optical signal is detectable either by observation or instrumentally and its response is related to the fluorescence or light intensity, distribution and lifetime.

The preparation of the compounds of formula (I), as such or in the form of pharmaceutically acceptable salts, represents a further aspect of the invention. The compounds of the invention can be prepared for instance according to the methods described in the following sections and in the experimental part. A general teaching about the preparation of cyanine scaffold can be found in Mujumdar R. B. et al., Bioconjugate Chem. 1993, 4(2), 105-111, which relates to the synthesis and labeling of sulfoindocyanine dyes.

However, in some cases, because of the presence of different functional moieties such as carboxylic acid or amide groups in the cyanines of the present invention, the use of protecting groups may be necessary to direct the reactions on the desired functional group.

Generally, special attention is required when manipulating the cyanines at the strong pH and temperature conditions necessary to remove the protecting groups, since the stability of the polymethine scaffold can be compromised in some cases, with severe degradation of the dyes. Contrary to what was expected, the compounds of the invention have been found very stable at basic pH (i.e. at about pH 11-12) and none or negligible degradation has been observed during the removal of the protecting groups.

### Experimental Part

The invention and its particular embodiments described in the following part are only exemplary and not to be regarded as a limitation of the present invention: they show how the present invention can be carried out and are meant to be illustrative without limiting the scope of the invention.

### Materials and Equipment

All commercially available reagents used in the synthesis were obtained from Sigma Aldrich and TCI and used without further purification. The Reference compound (HF-1) was prepared as described in Mahalingam S. M. et al., Bioconjugate Chem 2018, 29, 3320-3331.

All the reactions were monitored by HPLC (Agilent mod. 1100/1200) and HPLC-MS (Agilent mod. 1260, Quadrupole LC/MS Mod. 6120) equipped with an absorption detector set at different wavelengths (Column: YMC-Triart Phenyl, 250×4.6 mm/S−5 μm/12 nm), eluents: 0.1% ammonium acetate and acetonitrile.

Flash chromatographic purifications were performed on an automated purification system (CombiFlash® Rf+, Teledyne ISCO), using pre-packed silica C18 cartridges (Biotage® SNAP or SFAR), generally eluting with water/acetonitrile gradient.

A dual-beam UV-VIS spectrophotometer (Lambda 40, Perkin Elmer) was used to determine the absorbance (Abs) of the compounds of the invention. Emission/excitation (Em/Ex) spectra were carried out on a spectrofluorometer (FluoroLog-3 1IHR-320, Horiba Jobin Yvon) equipped with an F-3018 integrating sphere accessory. The measurements were performed using an excitation wavelength at maximum absorbance of different dyes, and the sample was excited with a 450W Xenon Light Source. Detection was performed by photomultiplier tubes (PMT-NIR) cooled detector or by TBX-04 detector. Dye solutions were carefully prepared to have an absorbance lower than 0.1 (optical densities) to minimize re-absorption phenomena.

Human colon adenocarcinoma HT-29 cells (ATCC) used for the cellular binding experiments were cultured in McCoy's 5A medium (Sigma-Aldrich), supplemented with 10% HyClone Fetal Clone III (Euroclone), 2 mM L-glutamine (Sigma-Aldrich), 100 IU/mL penicillin, 0.1 mg/mL streptomycin, 0.25 μg/mL amphotericin B (Antibiotic-Antimycotic solution, Life Technologies) and cells were grown at 37° C. in humidified atmosphere enriched with 5% CO₂. DPBS without MgCl₂ and CaCl₂ (Sigma-Aldrich) was used for cell rinsing.

### LIST OF ABBREVIATIONS

DCM Dichloromethane
DMF N,N-Dimethylformamide
TEA Triethylamine
HPLC High performance liquid chromatography
NMR Nuclear magnetic resonance
tR Retention time (HPLC)
RT Room temperature
CV Column volume
DMSO Dimethyl sulfoxide
PBS Phosphate buffered saline
C8-AZA 8-amino-N-(5-sulfamoyl-1,3,4-thiadiazol-2-yl)octanamide
NMM N-methylmorpholine
HBTU O-Benzotriazol-1-yl-N,N,N′,N′-tetramethyluronium hexafluorophosphate
HATU 1-[Bis(dimethylamino)methylene]-1H-1,2,3-triazolo[4,5-b]pyridinium 3-oxid hexafluorophosphate
TBTU 2-(1H-Benzotriazole-1-yl)-1,1,3,3-tetramethyluronium tetrafluoroborate
TSTU O—(N-Succinimidyl)-1,1,3,3-tetramethyluronium tetrafluoroborate
COMU (1-Cyano-2-ethoxy-2-oxoethylidenaminooxy)dimethylamino-morpholino-carbenium hexafluorophosphate

### Example 1: Synthesis of Compound 1

### Preparation of the Intermediate Compound (13)

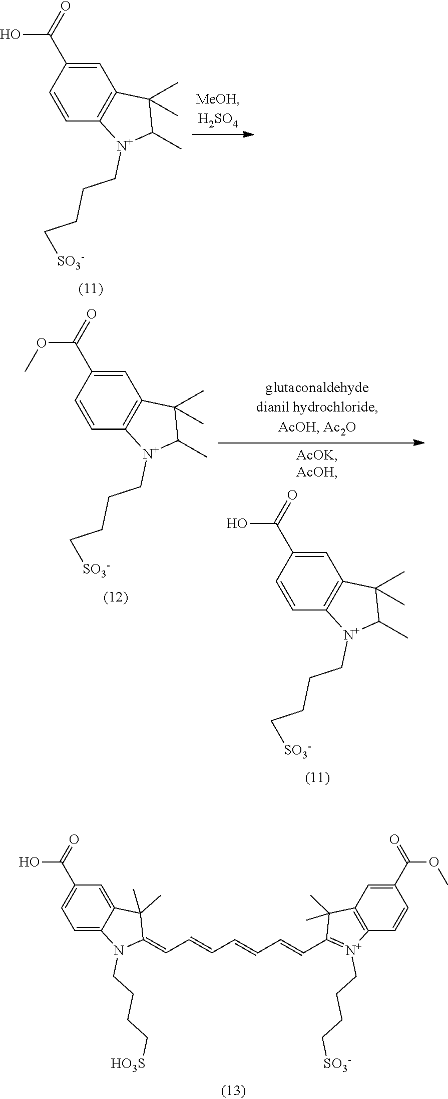

To a solution of Compound 11 (502 mg, 1.478 mmol), prepared as described in Example 2, in methanol (7.8 mL) conc. H₂SO₄ (0.810 mL) was added and the solution was refluxed for 2 hours. The mixture was cooled down to RT and concentrated under reduced pressure, diluted with water (pH 2) and purified by flash chromatography on a pre-packed C18 silica column (Biotage© SFAR 60 g) with a water-acetonitrile gradient (the desired compound was eluted with 28% acetonitrile). After freeze-drying, Compound 12 was obtained as a pale brown solid (447 mg, 86% yield, HPLC purity: 98.7% at 270 nm, MS: [M+H]⁺ 354.1).

Compound 12 (475 mg, 1.345 mmol) and glutaconaldehyde dianil hydrochloride (383 mg, 1.345 mmol) were dissolved in 4:1 acetic anhydride: glacial acetic acid mixture (10 mL) and heated for 1 hour at 120° C. in the dark. Then, Compound 11 (456 mg, 1.345 mmol) and potassium acetate (660 mg, 6.725 mmol) were added, diluting with other 10 mL of 4:1 acetic anhydride: glacial acetic acid mixture. After 30 minutes at 120° C., the reaction was stopped. The mixture was cooled down to RT and solvents were removed by reduced pressure. Cold diethyl ether (150 mL) was added, and the mixture was stirred for 1 hour in an ice bath. The precipitate was filtered under suction, dissolved in water/acetonitrile and purified twice by flash chromatography on a pre-packed C18 silica column (Biotage® SFAR 60 g) with a water-acetonitrile gradient (the cyanine was eluted with 30% acetonitrile). Fractions containing the pure product were combined, concentrated under reduced pressure and freeze-dried, obtaining 242 mg of a blue solid (24% yield, HPLC purity: 99.6% at 756 nm and 94.7% at 254 nm, MS: [M+H]⁺ 755.1).

### Preparation of the Intermediate Compound (15)

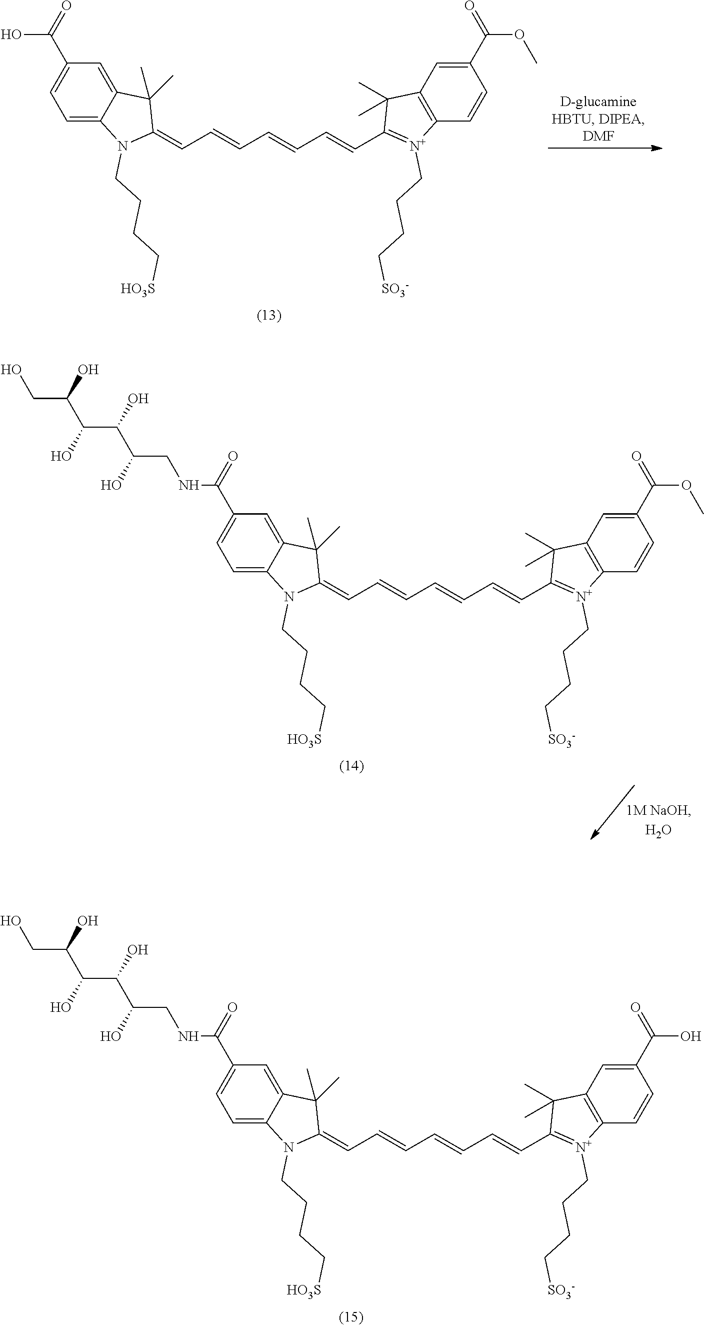

Compound 13 (242 mg, 0.321 mmol) was coupled with D-glucamine (70 mg, 0.385 mmol) in the presence of HBTU (146 mg, 0.385 mmol) and DIPEA (112 μL, 0.642 mmol) in DMF (13 mL), stirring at RT for 1 hour in the dark. Then, the majority of the solvent was removed under reduced pressure and the product was precipitated in cold ethyl acetate (60 mL), stirring for 1 hour in an ice bath. The precipitate was filtered under suction and used in the following step without further purification (313 mg, HPLC purity: 93.9% at 756 nm, MS: [M+H]⁺ 918.2).

Compound 14 (313 mg, 0.321 mmol theoretical) was dissolved in water (50 mL) and hydrolyzed at 40° C. for 7.5 hours maintaining pH=12 by continuous addition of 1 M NaOH (2.76 mL in total). The mixture was cooled down to room temperature, pH was adjusted to 7.5 with diluted HCl and the aqueous solution was purified by flash chromatography on a pre-packed C18 silica column (Biotage® SFAR 30 g) with a water/acetonitrile gradient (the cyanine was eluted with 15% acetonitrile). Fractions containing the pure product were combined, concentrated under reduced pressure and freeze-dried, obtaining 135 mg of a blue solid (35% yield over two steps, HPLC purity: 99.5% at 756 nm and 98% at 254 nm, MS: [M+H]⁺ 904.2).

### Preparation of 8-amino-N-(5-sulfamoyl-1,3,4-thiadiazol-2-yl)octanamide (C8-AZA)

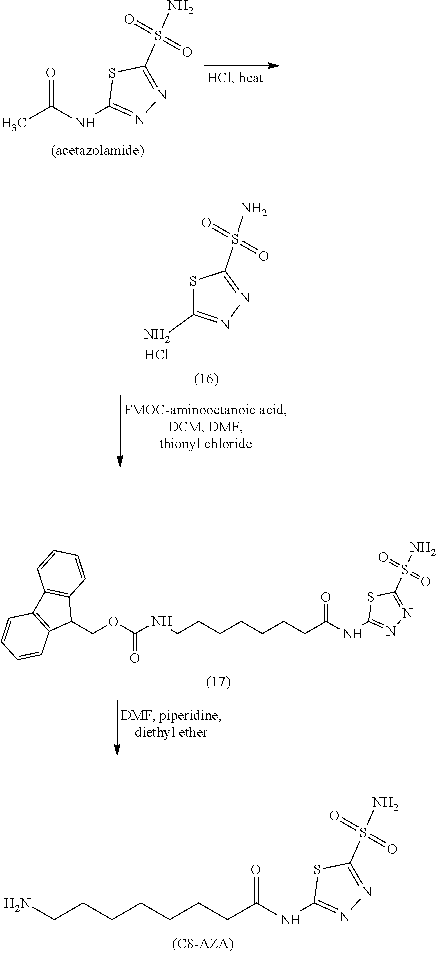 [SMILES: CC(=O)Nc1nnc(S(N)(=O)=O)s1 ; Nc1nnc(S(N)(=O)=O)s1 ; Cl ; NS(=O)(=O)c1nnc(NC(=O)CCCCCCCNC(=O)OCC2c3ccccc3-c3ccccc32)s1 ; NCCCCCCCC(=O)Nc1nnc(S(N)(=O)=O)s1]

In a round bottom flask acetazolamide (1103.56 mg, 4.96 mmol) was suspended in 1 M HCl (26 mL) and heated up to reflux for 1.5 hour, obtaining a clear solution. The mixture was concentrated under reduced pressure and purified by flash chromatography on a pre-packed C18 silica column (Biotage® Sfär C18 60 g) with a water-acetonitrile gradient (4 CV at 0% ACN, linear gradient from 0 to 100% ACN in 2 CV). Fractions containing the pure intermediate compound 16 thus obtained (eluted with 100% water) were combined, concentrated under reduced pressure and freeze-dried, obtaining a white solid (775.24 mg, 72% yield). HPLC purity: 97.8% at 270 nm, [M+H]⁺ 181.1.

In a round bottom flask cooled in an ice bath, Fmoc-8-aminooctanoic acid (500 mg, 1.311 mmol) was suspended in anhydrous DCM (30 mL) and a small amount of anhydrous DMF (20 μL, 0.262 mmol). Then, thionyl chloride (2.85 mL, 39.33 mmol) was slowly dropped. The mixture was stirred at room temperature for about 30 minutes, observing complete dissolution. Solvents and residual thionyl chloride were concentrated under reduced pressure. The yellow crude oil was immediately dissolved in a solution of hydrolyzed acetazolamide (compound 16) (283 mg, 1.311 mmol) and anhydrous pyridine (211 μL, 2.62 mmol) in anhydrous DMF (10 mL) and stirred at room temperature for 1.5 hour. The solution was then concentrated under reduced pressure and the product was precipitated with DCM. The white solid was filtered and dried under vacuum at 30° C. overnight (294.25 mg, 41% yield). HPLC purity: 93% at 270 nm and 254 nm, MS: [M+H]⁺ 544.2.

In a round bottom flask the so-isolated solid of compound 17 (294.25 mg, 0.541 mmol) was dissolved in anhydrous DMF (5.8 mL) and piperidine (1.16 mL) was added. After 30 min cold diethyl ether (100 mL) was added; the solid was then filtered, dissolved in methanol, the solution was concentrated to solid residue and treated with diethyl ether (100 mL). Finally, the pure product C8-AZA was recovered as a white powder which was dried under vacuum at room temperature overnight (163.56 mg, 94% yield). HPLC purity: 96.6% at 270 nm and 96.7% at 254 nm, MS: [M+H]⁺ 322.2.

### Synthesis of Compound 1

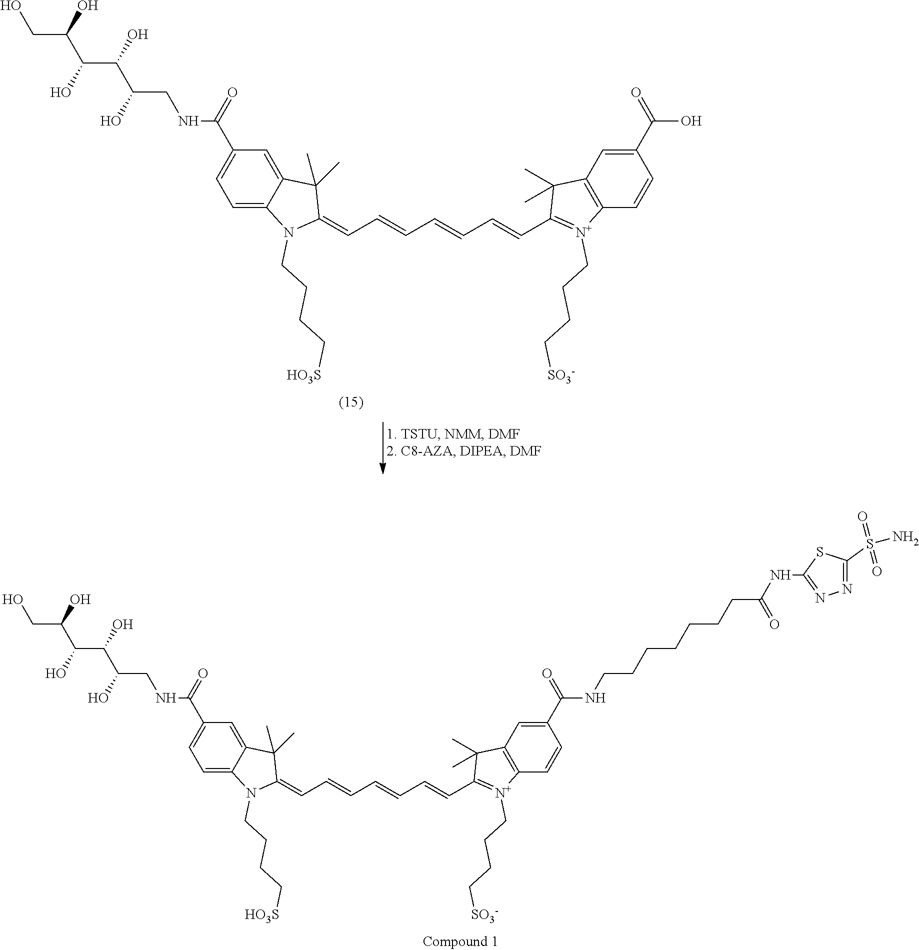

In a 50 mL centrifuge tube Compound 15 (15 mg, 0.016 mmol) was dissolved in dry DMF (1.5 mL) and NMM (4 μL, 0.036 mmol) and TSTU (7.5 mg, 0.024 mmol) were added. The dark blue/green solution was stirred for 1 hour at room temperature in the dark, reaching 88.1% of conversion at 756 nm. Cold ethyl acetate (25 mL) was added and the suspension was centrifuged for 10 min at 4010 rpm and 5° C. Then, the organic layer was decanted, the solid was suspended twice in ethyl acetate (10 mL) and centrifuged again. The crude solid was finally dried under nitrogen atmosphere and then dissolved in a solution of 8-amino-N-(5-sulfamoyl-1,3,4-thiadiazol-2-yl)octanamide (C8-AZA) (6.7 mg, 0.021 mmol) and DIPEA (12 μL, 0.066 mmol) in anhydrous DMF (2 mL). The solution was stirred in the dark at RT for 1 hour. Since the NHS ester was not completely converted into the desired product, an excess of C8-AZA (2.5 mg, 0.008 mmol) was added. The solution was stirred for other 30 minutes, then it was dropped into cold diethyl ether (25 mL). The precipitate was filtered under suction, the crude solid was dissolved in water/acetonitrile and purified by flash chromatography on a pre-packed C18 silica column (Biotage® SFAR 12 g) with a water-acetonitrile gradient (the cyanine was eluted with 25% acetonitrile). Fractions containing the pure product were combined, concentrated under reduced pressure and freeze-dried, obtaining 12.5 mg of a blue solid (62% yield, HPLC purity: 98.2% at 756 nm and 97.6% at 254 nm, MS: [M+H]⁺ 1207.2).

### Example 2: Synthesis of Compound 2

### Preparation of Intermediate Compound 19

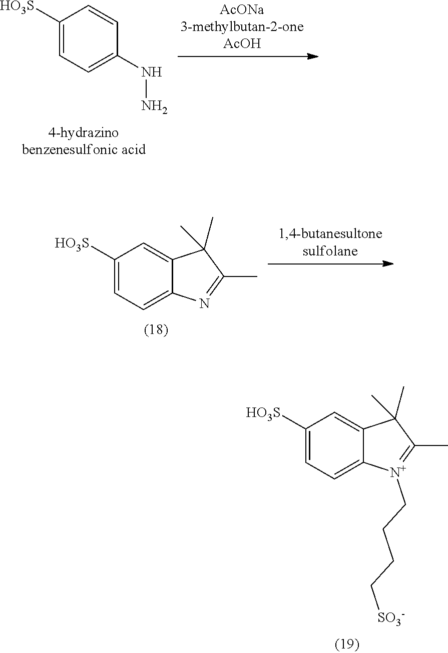

To a suspension of 4-hydrazinobenzenesulfonic acid (2.09 g, 0.011 mol) in glacial acetic acid (10 mL) 3-methylbutan-2-one (1.5 mL, 0.014 mol) and sodium acetate (1.64 g, 0.020 mol) were added. After stirring for 4.5 hours at 110° C. the orange solution was cooled to RT and precipitated in cold diisopropyl ether (100 mL). The solid was filtered under suction and dissolved with methanol together with SiO₂. Methanol was removed and the silica gel was loaded onto a silica gel column eluting with 30% methanol in ethyl acetate, giving Compound 18 as pale brown solid (2.46 g, 94% yield, HPLC purity: 90% 270 nm, MS: [M+H]⁺ 240.1).

A mixture of Compound 18 (1.32 g, 5.51 mmol) and 1,4-butanesultone (0.84 mL, 7.58 mmol) in sulfolane (2 mL) was heated at 120° C. for 24 hours. Then, the mixture was cooled down to RT and cold ethyl acetate (50 mL) was added, stirring for 1 hour in an ice bath. Then, the precipitate was filtered under suction and washed twice with cold ethyl acetate (2×50 mL). The crude product was dissolved in methanol, dried under vacuum and purified by flash chromatography on a pre-packed KP-Sil cartridge (Biotage® SNAP, 60 g) eluting with 50% methanol-ethyl acetate. Compound 19 was recovered as a pink solid (0.573 g, 28% yield, HPLC purity: 98.3% 270 nm, MS: [M+H]⁺ 376.0).

### Preparation of Intermediate Compound 11

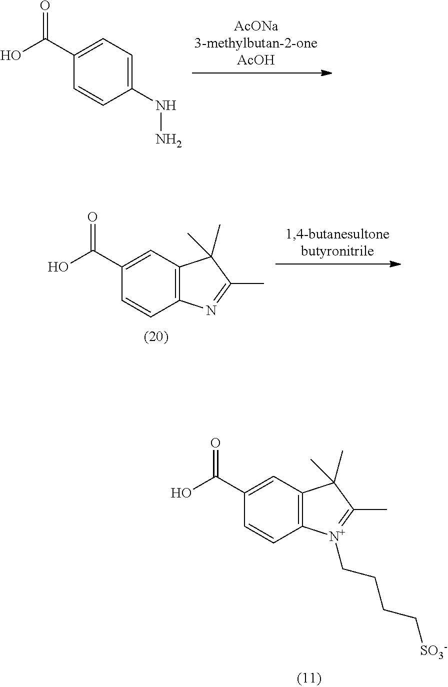

To a suspension of 4-hydrazinobenzoic acid (10 g, 0.053 mol) in glacial acetic acid (150 mL) 3-methylbutan-2-one (146 mL, 0.130 mol) and sodium acetate (11 g, 0.130 mol) were added. After stirring for 3 hours at 135° C. the brown solution was cooled to RT, concentrated under reduced pressure and suspended in 30 mL 9:1 water-methanol mixture. The solid (compound 20) was filtered under suction and washed with further 40 mL of 9:1 water-methanol, then dissolved in 200 mL sat. NaHCO₃ solution and extracted with dichloromethane (3×125 mL). The organic layer was concentrated under reduced pressure and the orange solid was dried in the oven for 18 h at 45° C., obtaining 9.4 g (87% yield, HPLC purity: 98% 270 nm, MS: [M+H]⁺ 204.8).

A mixture of Compound 20 (4.01 g, 19.7 mmol) and 1,4-butanesultone (2.4 mL, 23.5 mmol) in butyronitrile (4 mL) was heated at 120° C. for 54 hours. The mixture was cooled down to RT and cold acetone (100 mL) was added, stirring for 1 hour in an ice bath. Then, the precipitate was filtered under suction and washed twice with cold ethyl acetate (2×50 mL). The crude product was dissolved in 0.1% ammonium acetate aqueous solution and purified by flash chromatography on a pre-packed C18 silica column (Biotage© SNAP, 60 g), eluting the desired product with water, and removing by-products with acetonitrile. Desalting was performed on the same silica C18 column, loading the product dissolved in sodium acetate solution and washing with 0.05% formic acid aqueous solution (2 CV) and then water (2 CV). Fractions containing the desired product were concentrated under reduced pressure and freeze-dried, obtaining 4.8 g of a pink solid (72% yield, HPLC purity: 98% at 270 nm, MS: [M+H]⁺ 340.1).

### Preparation of Intermediate Compound 21

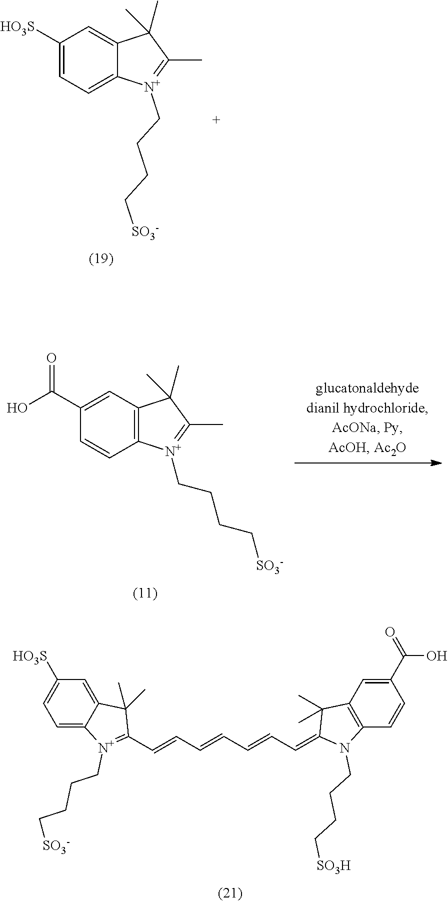

Compound 19 (100 mg, 0.266 mmol) and glucatonaldehyde dianil hydrochloride (75 mg, 0.266 mmol) were stirred at 50° C. for 1 hour in 2:1 acetic anhydride-glacial acetic acid mixture (15 mL) in the presence of sodium acetate (20 mg, 0.252 mmol). Then, Compound 11 (144 mg, 0.423 mmol) was added, followed by pyridine (0.36 mL, 4.520 mmol), and the dark mixture was heated at reflux in the dark for 4 hours. The green solution was cooled down to RT, and solvents were evaporated under reduced pressure obtaining a viscous residue. Methanol (2 mL) was added to dissolve part of the solid, that was then precipitated in cold diethyl ether (50 mL), stirring for 1 hour in an ice bath. The precipitate was filtered under suction, dissolved in water and purified three times on a pre-packed C18 silica column (Biotage® SFAR 30 g) with a water-acetonitrile gradient (Compound 19 was eluted with 12% acetonitrile). Finally, Compound 21 was freeze-dried, obtaining 84 mg of a green solid (40% yield, HPLC purity: 99% at 756 nm and 98% at 254 nm, MS: [M+H]⁺777.2).

### Synthesis of Compound 2 (by Activation with Standard Coupling Reagents)

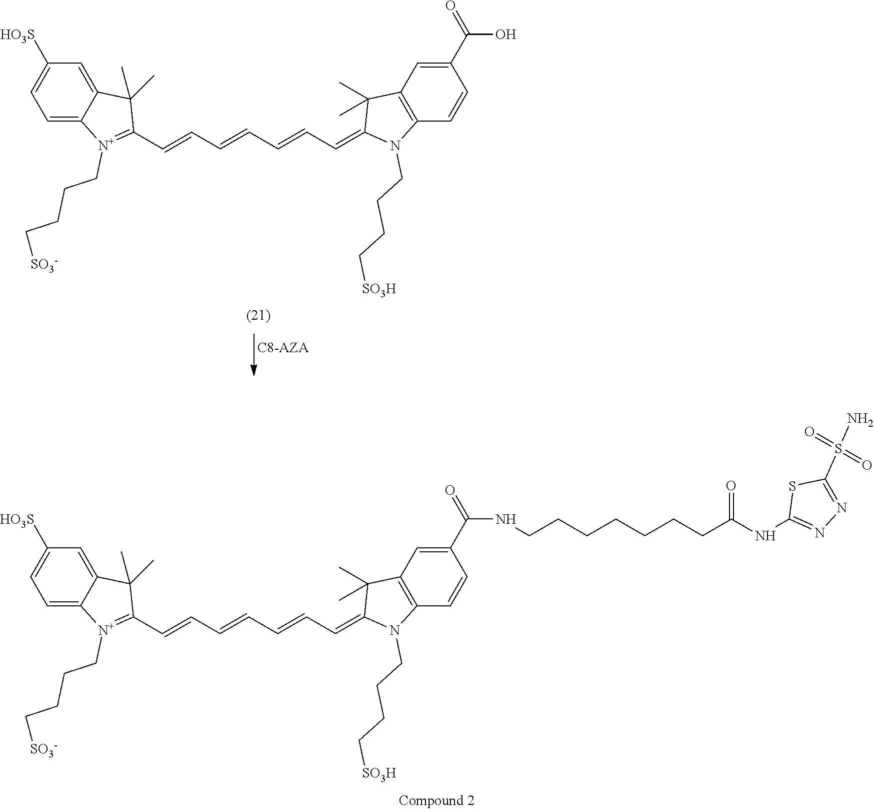

Compound 21 (9.8 mg, 0.0126 mmol) was dissolved in anhydrous DMF (4 mL), then COMU (8.1 mg, 0.0189 mmol), C8-AZA (6.1 mg, 0.0189 mmol), prepared as described in Example 1, and DIPEA (4.5 μL, 0.0252 mmol) were added. The dark green solution was stirred in the dark for 30 minutes at RT, then cold diethyl ether (50 mL) was added and the mixture was stirred in an ice bath for 30 minutes. The precipitate was filtered under suction, dissolved in water and purified by flash chromatography on a pre-packed C18 silica column (Biotage® SFAR 12 g) with a water-acetonitrile gradient (the cyanine was eluted with 25% acetonitrile). Fractions containing the pure product were concentrated under reduced pressure and freeze-dried, obtaining 7.1 mg of a green solid (52% yield, HPLC purity: 97.3% at 756 nm and 98.5% at 254 nm, MS: [M+H]⁺ 1080.2).

### Synthesis of Compound 2 (by Activation as NHS Ester)

Compound 21 (10.1 mg, 0.0129 mmol) was dissolved in anhydrous DMF (4 mL), then TSTU (4.9 mg, 0.0162 mmol) and NMM (3.5 μL, 0.032 mmol) were added. The dark green solution was stirred in the dark for 1 hour at RT. Cold ethyl acetate (40 mL) was then added and the mixture was stirred in an ice bath for 30 minutes. The precipitate was filtered under suction and washed with ethyl acetate (2×10 mL). The solid (ca. 90% of NHS active ester) was dissolved in anhydrous DMF (4 mL), then C8-AZA (6.2 mg, 0.019 mmol) and DIPEA (11 μL, 0.0645 mmol) were added and the solution was stirred in the dark for 1 hour. The product was precipitated by addition of cold diethyl ether (50 mL). The solid was filtered under suction, dissolved in water and purified by flash chromatography on a pre-packed C18 silica column (Biotage© SFAR 12 g) with a water-acetonitrile gradient (the cyanine was eluted with 25% acetonitrile). Fractions containing the pure product were concentrated under reduced pressure and freeze-dried, obtaining 11.5 mg of a green solid (82% yield, HPLC purity: 98.5% at 756 nm and 98.5% at 254 nm, MS: [M+H]⁺ 1080.2).

### Example 3: Synthesis of Compound 3

### Preparation of Intermediate Compound 22

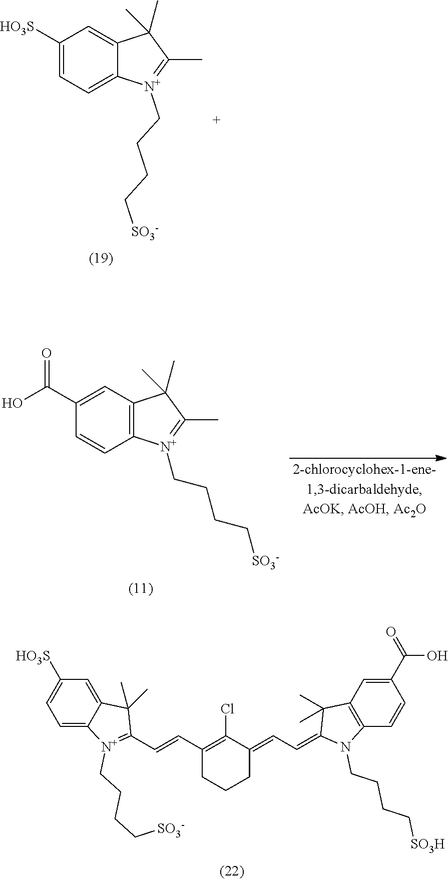

Compound 19 (100 mg, 0.266 mmol), Compound 11 (90 mg, 0.266 mmol) and potassium acetate (72 mg, 0.744 mmol) were dissolved in a 2:1 acetic anhydride: glacial acetic acid mixture (10 mL) at 50° C. Then, 2-chlorocyclohex-1-ene-1,3-dicarbaldehyde (55 mg, 0.319 mmol) was added and the mixture was heated at 100° C. in the dark for 1.5 hour. The mixture was cooled down to RT and solvents were removed under reduced pressure. The viscous residue was partially dissolved in methanol (2 mL) and precipitated in cold diethyl ether (70 mL) after stirring for 1 hour in an ice bath. The precipitate was filtered under suction, dissolved in water (pH 3), and purified by flash chromatography on a pre-packed C18 silica column (Biotage® SFAR 30 g) with a water-acetonitrile gradient (the cyanine was eluted with 25% acetonitrile). A second purification was performed at pH 8 on the same C18 silica column with a water-acetonitrile gradient (the cyanine was eluted with 18% acetonitrile). Compound 22 was finally freeze-dried, obtaining 63 mg of a green solid (28% yield, HPLC purity: 99% at 780 nm and 95% at 254 nm, MS: [M+H]⁺ 851.2).

### Synthesis of Compound 3

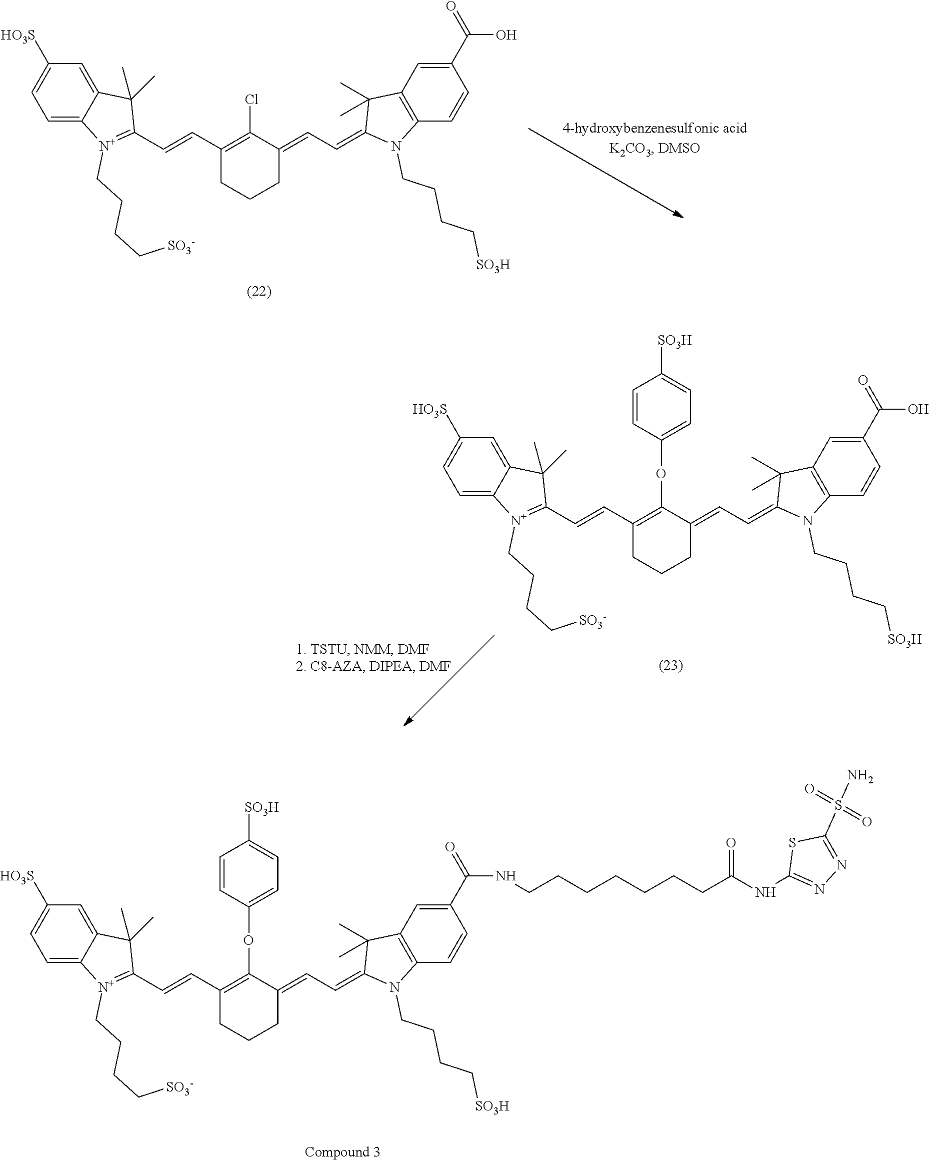

Compound 22 (57 mg, 0.067 mmol) was dissolved in DMSO (5 mL) and dropped into a suspension of 4-hydroxybenzenesulfonic acid (131 mg, 0.670 mmol) and potassium carbonate (92 mg, 0.670 mmol) in DMSO (5 mL). The mixture was stirred in the dark at RT overnight. Then, the mixture was precipitated in cold ethyl acetate (200 mL), stirring for 1 hour in an ice bath. The precipitate was filtered under suction, dissolved in water, quickly acidified at pH 4 with diluted HCl, and purified by flash chromatography on a pre-packed C18 silica column (Biotage® SFAR 12 g) with a water-acetonitrile gradient (the cyanine was eluted with 20% acetonitrile). Fractions containing the pure product were concentrated under reduced pressure and freeze-dried obtaining 58 mg of a green solid (88% yield, HPLC purity: 99% at 780 nm and 94% at 254 nm, MS: [M+H]⁺ 989.1).

In a 25 mL centrifuge tube Compound 23 (14.3 mg, 0.015 mmol) was dissolved in anhydrous DMF (2 mL), then NMM (4 μL, 0.033 mmol) and TSTU (7 mg, 0.023 mmol) were added. The dark green solution was stirred for 1 hour at RT, obtaining 92.5% of conversion at 780 nm. Cold ethyl acetate (20 mL) was added and the suspension was centrifuged for 10 min at 4010 rpm and 5° C. Then, the organic layer was decanted, the solid was suspended twice in ethyl acetate (10 mL) and centrifuged again. The crude solid was finally dried under nitrogen atmosphere and then dissolved in a solution of C8-AZA (6.1 mg, 0.019 mmol) and DIPEA (13 μL, 0.075 mmol) in anhydrous DMF (2 mL). The solution was stirred in the dark at RT for 1 hour, but since some NHS ester was still present, an excess of C8-AZA (2.4 mg, 0.0075 mmol) was added. The reaction was stirred in the dark at RT overnight. Cold ethyl acetate (25 mL) was then added and the precipitate was filtered under suction. The crude material was dissolved in water and acetonitrile (pH adjusted to 7.5) and purified twice by flash chromatography on a pre-packed C18 silica column (Biotage® SFAR 12 g) with a water-acetonitrile gradient (the cyanine was eluted with 28% acetonitrile). The pure product was freeze-dried obtaining 7.9 mg of a green solid (42% yield, HPLC purity: 98.9% at 780 nm and 97.9% at 254 nm, MS: [M+H]⁺ 1292.1).

### Example 4: Optical Characterization

The compounds of the invention were characterized in terms of their optical properties in vitro in aqueous medium (i.e., water/PBS pH 7.4) and in a clinical chemistry control serum (Seronorm, Sero S A), mimicking the chemical composition and optical properties of human serum. All solutions were freshly prepared. In particular, the compounds of the invention are characterized by absorption maxima comprised in the range from about 750 nm to 780 nm.

The excitation and emission maxima in PBS and Seronorm and the molar extinction coefficient (E) in water of representative compounds of formula (I) are shown in Table I in comparison with the known CA-IX targeting fluorescent probe named Hypoxyfluor-1 (HF-1, for instance disclosed in Bioconjugate Chem 2018, 29, 3320-3331). The compounds of the invention displayed a red shift in absorption and emission in Seronorm with respect to PBS due to some interaction of the probes with serum proteins. Moreover, the extinction coefficients were higher than 200,000 mM⁻¹ cm⁻¹ for all compounds.

TABLE I
Excitation/Emission maxima and molar extinction coefficient
(ε) of representative compounds of formula (I)

| | Max Ex/Em | Max Ex/Em | ε |
|---|---|---|---|
| | (nm, PBS pH 7.4) | (nm, Seronorm) | (mM⁻¹cm⁻¹) |
| HF-1 (Reference) | 772/798 | 792/808 | 255,000 |
| Compound 1 | 752/783 | 768/791 | 240,000 |
| Compound 2 | 754/784 | 768/788 | 260,000 |
| Compound 3 | 776/802 | 792/810 | 240,000 |

### Example 5: Receptor Binding Affinity

### Affinity to Carbonic Anhydrase Catalytic Site

The binding affinity of the compounds of formula (I) to Carbonic Anhydrase (CA) catalytic site was determined to assess the targeting efficacy of the molecular vectors, even after labeling the targeting ligand with the fluorescent portion of the probe.

In detail, the receptor affinity of representative probes bearing the acetazolamide moiety as targeting ligand to a CA catalytic site was determined by testing their binding to a recombinant bovine CAII enzyme (bCAII). Such enzyme in fact is highly available and has a catalytic site with high degree of homology in different carbonic anhydrase isoforms (Aggarwal M. et al., J Enzyme Inhib Med Chem 2013; 28(2): 267-277).

The dissociation constant (Ko) of compounds of formula (I) for bCAII was estimated by performing a fluorescence-based assay, exploiting the drop of fluorescence deriving from the displacement of carbonic anhydrase-bound dansylamide (DNSA) in presence of competitors and was adapted for application in 96-well plates from Wang S C et al., Biochem Mol Biol Educ. 2006, 34(5): 364-368. This assay is based on the fluorescence emission spectrum of DNSA, which displays differential peaks accordingly to the state of the molecule: the emission is at about 570 nm when DNSA is free in solution, whereas is shifted to 460 nm when DNSA is bound to CA. In both cases DNSA is excited at about 320 nm. Thus, excitation of carbonic anhydrase aromatic residues (Trp) at 285 nm generates an emission at 340 nm, that is quenched in presence of bound DNSA. The quenching occurs by FRET (fluorescence resonance energy transfer) to bound DNSA, which is in turn excited, emitting at 460 nm.

Unbound DNSA, which is not in proximity of the excited Trp, is unaffected by this phenomenon and does not generate any background disturbing fluorescence. Molecules that bind to CA in the same binding site of DNSA, such as sulfonamides or derivatives, competitively displace DNSA from CA. The degree of displacement is function of the affinity of competitors and can be followed through the drop of fluorescence at 460 nm.

In detail, 5-(Dimethylamino)-1-naphthalenesulfonamide (dansylamide, DNSA, Sigma-Aldrich 218898) was reconstituted in 20 mM Tris pH 8, aliquoted and stored at −20° C.; bCAII (recombinant, expressed in E. Coli, Sigma-Aldrich) was divided in aliquots and stored at −20° C. Scalar concentrations of compounds of formula (I) (range 0-9.5 μM) and acetazolamide, as internal control (range 0-22.5 μM), were incubated with 0.25 μM bCAII and 5 μM DNSA in 20 mM Tris pH 8.

DNSA fluorescence emission at 462 nm from each well was recorded after excitation at 285 nm (Trp wavelength of excitation) and consequent selective excitation of DNSA bound to bCAII by FRET. Fractional saturation of bCAII was experimentally determined through normalization of fluorescence data between 1 (no compounds) and 0 (maximal DNSA displacement by presence of binding competitors), according to the equation below:

$$r = \frac{{FL}_{obs} - {FL}_{min}}{{FL}_{max} - {LF}_{min}}$$

where r is fractional saturation, FL_{obs} is the recorded fluorescence intensity, FLₘᵢₙ is the fluorescence intensity recorded at maximal concentration of compounds, and FLₘₐₓ is fluorescence recorded in absence of compounds.

The K_{D} of CA-binding compounds (=KD_{comp}) was then derived by mathematical fitting of experimental data according to the equation:

$$r = \frac{1}{1 + \frac{{KD}_{DNSA}}{[ DNSA ]} * ( 1 + \frac{[ comp ]}{{KD}_{comp}} )}$$

where r is fractional saturation (calculated from experimental data as described above), [DNSA] is DNSA nanomolar concentration, KD_{DNSA} is the dissociation constant at equilibrium of DNSA (previously experimentally determined=169.6 nM), and [comp] is the nanomolar concentration of the CA-targeting compounds under analysis. Mathematical fitting was performed using Microsoft Excel Solver. Acetazolamide was included in every test as internal control of the assay (acetazolamide K_{D}=29.6±2.6 nM, previously estimated).

All compounds were tested in triplicate. The results of the assays performed are summarized in Table II, which reports, for comparison purposes, the corresponding results obtained for the unlabeled acetazolamide and for the known carbonic anhydrase targeting agent Hypoxyfluor-1 (HF-1).

The tested probes showed a very high affinity to the carbonic anhydrase receptor, displaying K_{D} values lower than 7 nM, about 5-7 times lower than acetazolamide and 1.5-2.5 times lower than HF-1.

TABLE II
Binding affinity to the CAII receptor of representative
compounds of formula (I) compared to the unlabeled
acetazolamide and to known probe HF-1.

| | CAII receptor affinity |
|---|---|
| | (K_{D}, nM) |
| Acetazolamide (Ref.) | 29.6 ± 2.6 |
| HF-1 (Ref.) | 9.5 ± 1.0 |
| Compound 1 | 6.2 ± 0.6 |
| Compound 2 | 3.7 ± 0.3 |
| Compound 3 | 3.8 ± 0.5 |

### Cellular Binding Assessment

To further confirm these results showing improved binding affinity, the binding of the probes to CA-IX was assessed by using an in vitro model with the human colorectal adenocarcinoma cell line HT-29 (ATCC). In fact, by both western blot and flow cytometry, HT-29 cells were demonstrated to constitutively express high levels of CA-IX under normoxic conditions. Indeed, by using the anti-human CA-IX antibody MAB2188 (R&D Systems) and the DAKO QUIFIKIT, according to the instruction of the datasheet, HT-29 cells were estimated to express about 150,000 CA-IX molecules per cell. Thus, HT-29 cells were used to measure the binding of all compounds to CA-IX, expressed in its physiological environment at the cell-surface. Briefly, HT-29 cells were detached with StemPro® Accutase® Cell Dissociation Reagent (Life Technologies), collected in DPBS and counted. At least 2·10⁵ cells were placed in 1.5 mL tubes on ice and resuspended in 50-100 μL of cold FACS buffer (eBioscience™ Flow Cytometry Staining Buffer, Invitrogen) containing scalar concentrations of compounds (compound 1: range 1-5 μM; compounds 2 and 3: range 0-20 μM). After incubation (1 hour and 30 min on ice in the dark), cells were rinsed 3 times in 300 μL of cold FACS buffer by centrifugation (5 min, 4° C., 350 RCF), discarding the supernatant. Cell pellets were then resuspended in 200 μL of cold FACS buffer (or in a suitable volume in order to obtain no more than 1000 recorded events per μL during FACS analysis) and analyzed with the flow cytometer Accuri™ C6 (BD Biosciences). The mean cell-associated fluorescence was plotted against concentration and the dissociation constant at equilibrium (Ko) was inferred by mathematical fitting with GraphPad Prism v.9 software, according to the following equation:

$$Y = \frac{{B}_{max} * X}{X + {K}_{D}} + NS * X + background$$

where Bₘₐₓ is the maximum specific binding in the same units as Y.
K_{D} is the equilibrium dissociation constant, in the same units as X.
NS is the slope of nonspecific binding in Y units divided by X units.
Background is the amount of nonspecific binding with no added Compound.

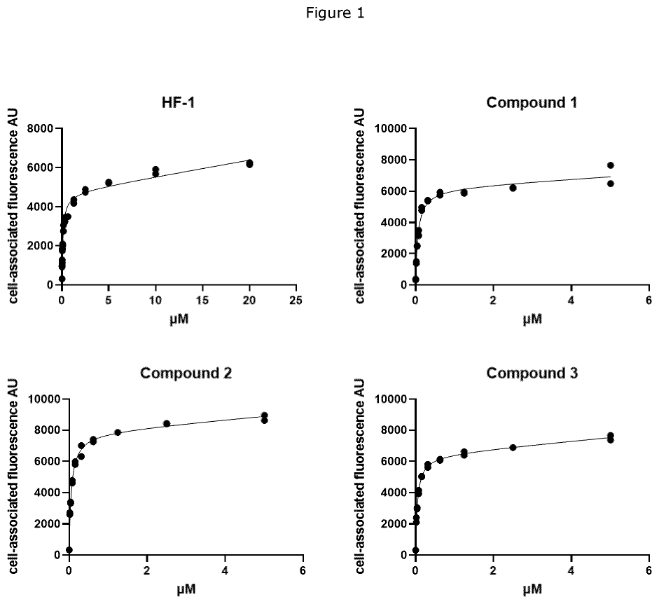

All compounds were analyzed in at least three independent experiments. The calculated K_{D} are summarized in Table III and compared with the one obtained for HF-1. Binding curves are shown in FIG. 1.

TABLE III
Binding affinity to CA-IX expressing HT-29 cells of representative
compounds of formula (I) in comparison to HF-1.

| | CA-IX receptor affinity |
|---|---|
| | (K_{D}, nM to HT-29 cells) |
| HF-1 (Ref.) | 115 ± 8 |
| Compound 1 | 68 ± 4 |
| Compound 2 | 54 ± 3 |
| Compound 3 | 62 ± 4 |

This assay confirmed that compounds of the invention bearing a C8-AZA moiety are endowed of a high affinity to CA-IX expressing cells, with K_{D} lower that 70 nM, and show a remarkable improvement in binding features compared to HF-1, which bears the same moiety but in the meso (central) position of the cyanine scaffold.

### Cellular Specificity

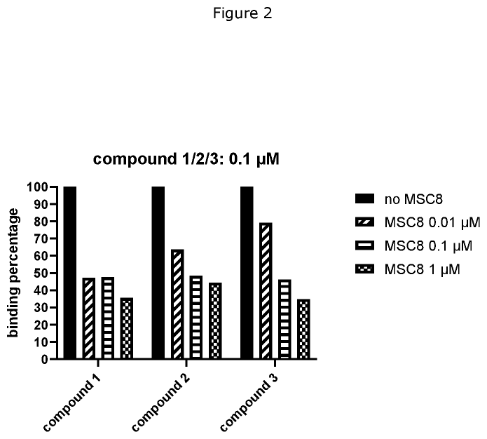

For all compounds, additional in vitro experiments were conducted to assess the CA-IX targeting specificity, by using the fully human recombinant MSC8 antibody, which has been demonstrated to selectively recognize the isoform IX of human CAs and block CA-IX enzymatic activity (WO2011/139375). HT-29 cells were co-incubated, on ice in suspension, with compounds 1/2/3, at the established concentration of 0.1 μM, alone or in combination with different concentrations of MSC8 (0.1×, 1×, and 10× with respect to compounds). The resulting binding of compound 1/2/3 was then recorded as cell-associated fluorescence intensity and expressed as percentage respect to the cell-associated fluorescence resulting from samples incubated with compound 1/2/3 in absence of MSC8, taken as 100%. Indeed, MSC8 reduced in a dose-dependent manner the binding of all compounds to HT-29 cells, indicating that it prevented their access to the catalytic pocket of CA-IX, therefore confirming their specificity for CA-IX targetingResults are shown in FIG. 2.

### Example 6: Cell Uptake

The HT-29 cells were used as in vitro model to assess the cell uptake of the CA-IX targeting compounds of the invention, more specifically whether cellular internalization of compounds occurred after targeting to cell-surface-expressed CA-IX. Cellular uptake was evaluated by flow cytometry. Briefly, the compounds were diluted to the working concentration of 1 μM in serum-free medium, supplemented with 25 mM HEPES pH 7.4; in competition experiments, a 100× excess of acetazolamide (Sigma-Aldrich A6011, dissolved in DMSO at 10 mg/mL stock concentration and stored at −20° C. in aliquots) was added in the same treatment medium. Treatment with unconjugated dyes were used to estimate non-specific CA-IX-independent cellular uptake. Cell incubation on ice was used to discriminate the cell-associated fluorescence deriving from internalized and cell-surface associated compounds (37° C.), from the one deriving from solely cell surface-bound compounds (on ice). Treatment of HT-29 cells, grown in 24-well plates, was performed by rinsing cells in DPBS and then by incubation with treatment solutions, for the indicated times and temperatures, in the dark. At the end of treatment, cells were rinsed 3 times with cold DPBS, and detached from the plate with StemPro® Accutase® Cell Dissociation Reagent, collected in 1.5 mL tubes after dilution in DPBS and centrifuged (5 minutes, 350 RCF, 4° C.). The supernatant was then discarded, and the cell pellet was resuspended in 300 μL of cold FACS buffer, or in a suitable volume in order to obtain no more than 1,000 recorded events per μL during FACS analysis. Samples were analyzed with the flow cytometer Accuri™ C6 (BD Biosciences) using the 640 nm excitation laser and the emission 780/60 nm filter. At least 15000 valid events were collected for each sample. At the end of analysis, each sample was plotted in histograms, being X the fluorescence intensity (FL4-A) in log scale and Y the counts. Usually, a gaussian distribution of the cell population was observed. The data retrieved were the mean fluorescence of each population, considered the fluorescence intensity value of the corresponding sample. Independent replicates were performed for each experimental condition. Normalized cell-associated fluorescence was calculated with the following formula:

$$Normalized FL = \frac{( FL treated - FL untreated )}{FL untreated}$$

In experiments where the effect of an excess of acetazolamide had to be compared with the standard uptake condition (i.e., in serum-free medium), data were expressed as percentage of the uptake in standard conditions.

To evaluate the time dependent uptake, the experiments were conducted from 1 to 6 hours of cell treatment with a compound concentration of 1 μM, both on ice (internalization blocked) and at 37° C. (internalization-permissive temperature) in order to differentiate cell-associated fluorescence deriving from internalized compounds from that due to cell surface-associated one. The cell surface-associated fluorescence remained roughly constant over time while the binding of the corresponding unconjugated dyes was undetectable. On the contrary, for compounds 1, 2 and 3 of the invention the total cell-associated fluorescence increased over time, indicating their progressive internalization. The internalization potential was calculated as percentage of internalized compounds after 2 hours of cell treatment (arbitrarily chosen) at 37° C.

These results are reported in Table IV together with the slope of the fitting line relating to the cell-associated fluorescence increase, which may be considered representative of the accumulation tendency of the different compounds.

TABLE IV
Internalization potential of representative compounds of formula (I)

| | % internalization | Slope of cell-associated fluorescence |
|---|---|---|
| | (mean ± std. dev.) | increase (37° C., 1-6 hours) |
| Compound 1 | 22.8 ± 8.2 | 1.0 |
| Compound 2 | 40.8 ± 9.9 | 1.9 |
| Compound 3 | 49.2 ± 3.9 | 2.7 |

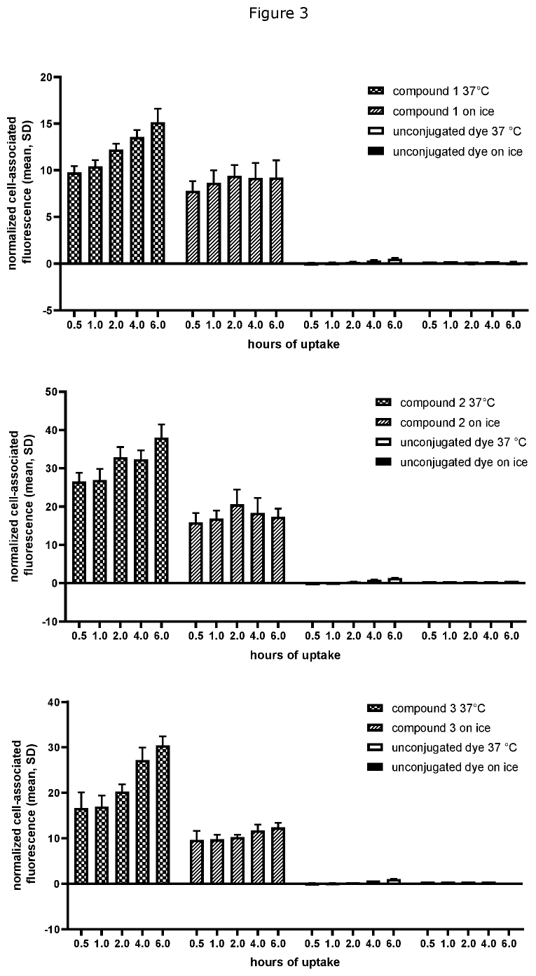

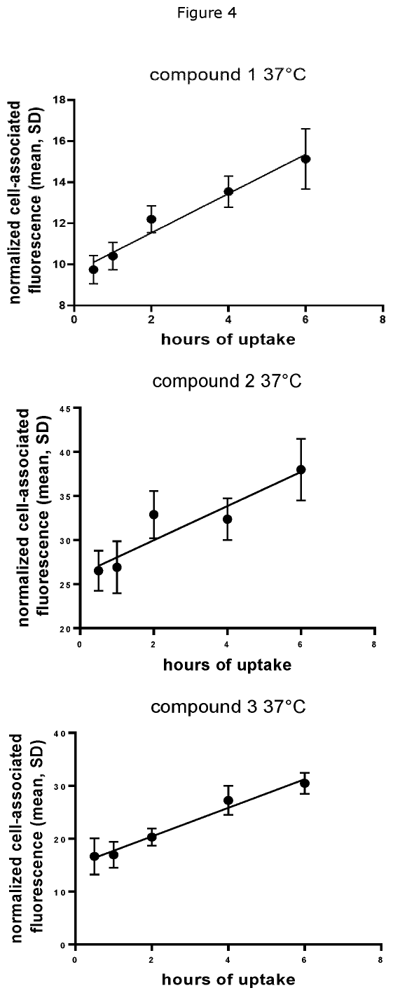

Moreover, FIGS. 3 and 4 report respectively the time-dependent uptake of compounds 1, 2 and 3 and of the corresponding unconjugated dyes, at 37° C. and after incubation on ice (FIG. 3) and a linear representation of cellular uptake of compounds 1, 2, 3 at 37° C. (FIG. 4).

To analyze the receptor-mediated uptake, i.e. whether the uptake of the probes was mediated by binding to the target receptor CA-IX, competition experiments were conducted. Compounds were administered to cells (1 μM, 2 hours at 37° C.) in presence or absence of 100× excess of acetazolamide, competing with the co-administered compounds for CA-IX binding site. In the above conditions, the cell-associated fluorescence for each probe decreased to the values reported in Table V, indicating that most of the recorded cellular uptake relied on CA-IX targeting.

TABLE V
Residual cellular uptake of representative
compounds of formula (I)

| | % uptake in presence of 100x excess of acetazolamide |
|---|---|
| Compound 1 | 16.4 ± 1.9 |
| Compound 2 | 28.4 ± 2.2 |
| Compound 3 | 18.1 ± 11.5 |

### Example 7: Tumor Imaging Efficacy

To evaluate the in vivo efficacy of the compounds of the invention, a hypoxic HT-29 colorectal tumor, subcutaneously induced, was used as an animal model due to the well-known hypoxic tumor microenvironment. HT-29 cells were collected and washed once with serum-free medium. Five million HT-29 cells were suspended in 0.1 mL serum-free medium and injected subcutaneously in the right flank of 5 weeks old mice. Tumor development was followed by caliper measurement twice a week, from 7 days after inoculation. Tumor volume was calculated according to the formula: (L*W2)/2, where L and W are the maximum length and width of the tumor.

Optical Imaging experiments on HT-29 tumor bearing animals were performed after administration of HF-1, as reference compound, and compounds 1, 2 and 3, all at a dose of 1 nmol/mouse in an administration volume of 0.1 mL (corresponding to about 5 mL/kg, considering a 20 g-mouse) at an injection rate of about 1 mL/min, by the IVIS Spectrum system when the tumor reached at least 200 mm³ volume.

For each group, in vivo OI experiments were performed at 0.5, 1, 2, 3, 4, 6 and 24 hours after intravenous administration of the fluorescent compounds. During imaging experiments animals were maintained under gaseous anesthesia. Regions of interest (ROIs) were drawn on the tumor and on a reference background healthy region (hind limb muscle) of the mouse for each fluorescence image at every time point to evaluate signal intensity in the tissues (expressed as Average Radiant Efficiency). The ratio between the fluorescence signal in the tumor and in the background healthy tissue (tumor-to-background ratio, TBR) was then calculated to assess the contrast.

The results are displayed in FIG. 5 and in Table VI, reporting the values of in vivo TBR from samples collected at 24 hours after administration. All the compounds of the invention and HF-1 were tested at a dose of 1 nmol/mouse.

The compounds of the invention showed selective accumulation in the tumor and values of TBR from 1.2 to 3.2 times higher than the reference compound HF-1 at the same dose of 1 nmol/mouse.

TABLE VI
Values of in vivo TBR (mean ±
SD) at 24 hours after administration

| | TBR (mean ± SD) |
|---|---|
| HF-1 (1 nmol) | 1.8 ± 0.3 |
| Compound 1 | 5.9 ± 1.2 |
| Compound 2 | 5.1 ± 1.9 |
| Compound 3 | 2.2 ± 0.6 |

### Example 8: Biodistribution and Elimination

The ex vivo biodistribution of the representative compounds 1, 2 and 3 and of the reference compound HF-1 (at 1 nmol/mouse) was evaluated in HT-29 excised tumors and tissues at the end of the in vivo OI sessions 24 hours post intravenous administration. At the end of the in vivo experiment, animals were euthanized by overdose of gaseous anesthesia followed by cervical dislocation. Tumor tissue, muscle, and other organs of interest were removed for ex vivo fluorescent signal measurements. The fluorescence signal intensity was measured by the IVIS Spectrum system. Regions of interest (ROIs) were drawn around each organ and tumor using the Living Image Software (PerkinElmer) and evaluating signal intensity in the tissues (expressed as Average Radiant Efficiency). The ratio between the fluorescence signal in the organs and tumor was then calculated to assess the contrast to hind limb muscle (representing the background).

The results are displayed in FIG. 6 (a) and in Table VII wherein it is evidenced that for the compounds of the invention, injected at the dose of 1 nmol/mouse, the fluorescence signal observed in tumor regions was remarkably higher compared to the fluorescence signal of the background (hind limb muscle), with tumor-to-background values higher than about 6.

Conversely, it is to be noted that the reference compound HF-1 at the same dose of 1 nmol/mouse showed much lower difference between fluorescent signals in tumor and in muscle (background).

TABLE VII
Values of ex vivo TBR (mean ± SD) from samples
collected 24 hours after administration of compounds
1, 2 and 3 and of reference compound HF-1.

| | HF-1 | Comp 1 | Comp 2 | Comp 3 |
|---|---|---|---|---|
| TBR ± SD | (1 nmol) | (1 nmol) | (1 nmol) | (1 nmol) |
| HT-29 tumor | 3.1 ± 0.3 | 16.4 ± 4.4 | 15.2 ± 5.9 | 5.8 ± 2.2 |

Moreover, FIG. 6 (b) and Table VIII show the accumulation and distribution of the fluorescent probes in the dissected organs, reporting the ex vivo values of organs-to-muscle ratio from samples collected at 24 hours after the administration of representative compounds 1, 2 and 3 and of the reference compound HF-1 (1 nmol/mouse).

Compounds fluorescence showed a very low concentration in most tissues, except for kidneys (probably due to renal excretion of unbound dyes) and liver. A certain uptake can be also seen in the stomach, which is known to express CA-IX.

TABLE VIII
Values of organ-to-muscle ratio (mean ±
SD) 24 hours after administration of compounds
1, 2 and 3 and of reference compound HF-1.

| Organ-to- | HF-1 | Comp 1 | Comp 2 | Comp 3 |
|---|---|---|---|---|
| muscle ratio | (1 nmol) | (1 nmol) | (1 nmol) | (1 nmol) |
| intestine | 2.2 ± 1.3 | 1.5 ± 0.3 | 1.2 ± 0.1 | 1.5 ± 0.2 |
| kidney | 2.4 ± 0.3 | 8.3 ± 1.9 | 4.0 ± 1.7 | 4.7 ± 1.6 |
| liver | 5.7 ± 0.2 | 3.3 ± 1.1 | 2.7 ± 0.4 | 3.1 ± 0.6 |
| skin | 1.4 ± 0.0 | 1.1 ± 0.2 | 1.6 ± 0.4 | 1.0 ± 0.1 |
| spleen | 1.4 ± 0.3 | 0.5 ± 0.1 | 1.0 ± 0.1 | 1.0 ± 0.1 |
| stomach | 4.0 ± 1.7 | 11.3 ± 3.2 | 7.1 ± 1.2 | 3.5 ± 0.3 |

### REFERENCES

1. Onda N. et al., Int J Cancer 2016, 139:673-682
2. Tummers Q. et al., PlosOne 2015 (doi.org/10.1371/journal.pone.0129766)
3. Gioux S. et al., Mol Imaging 2010, 9(5): 237-255
4. Bunschoten A. et al., Bioconjugate Chem. 2016, 27:1253-1258
5. Hilvo M. et al., J Biol Chem 2008; 283(41): 27799-27809
6. Wykoff et al., Cancer Res. 2000, 60:7075-7083
7. Muz et al., Hypoxia (Auckl) 2015; 3:83-92
8. WO2014/128258
9. McDonald et al., Am J Clin Oncol 2020; 43(7): 484-490
10. Wichert et al., Nat Chem 2015; 7(3): 241-249
11. Groves et al., Bioorg Med Chem Lett 2012; 22:653-657
12. WO2017/161195
13. Mahalingam S. M. et al., Bioconjugate Chem 2018, 29:3320-3331
14. Aggarwal M. et al., J Enzyme Inhib Med Chem 2013, 28(2): 267-277
15. Wang S C et al., Biochem Mol Biol Educ. 2006, 34(5): 364-368
16. WO2011/139375

1. A compound of formula (I), or a pharmaceutically acceptable salt thereof, 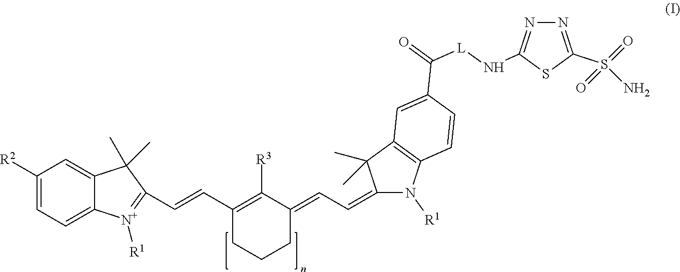
wherein
each R¹ is independently a straight or branched C₁-C₁₀ alkyl substituted by a group —SO₃H;
R² is selected from —SO₃H, a group —CONH—Y wherein Y is a straight or branched C₁-C₁₀ alkyl substituted with at least two hydroxyl groups, and a group (II) 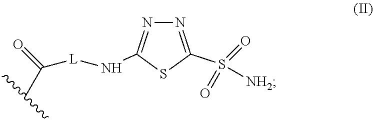
R³ is selected from hydrogen and a group phenyl or —O-phenyl optionally substituted with —SO₃H;
L is a bond or a linker;
n is an integer equal to 0 or 1.

2. The compound of formula (I) according to claim 1, wherein L is a group of formula —NH—(CH₂)ₚ—CO— or a diradical of one or more moiety selected from the group consisting of an amino acid; a peptide comprising from 2 to 10 amino acids in L or D configuration; 4-aminomethylbenzoic acid; cysteic acid; a polyethylene glycol; amino-polyethylene glycol-carboxylic acid; diaminobutyric acid; and diaminopropionic acid; or it is a group -L₁-L₂- wherein L₁ is a diradical of a diamine; and L₂ is a diradical of a dicarboxylic acid; wherein p is an integer comprised between 1 and 20.

3. The compound of formula (I) according to claim 1, wherein L is a group —NH—(CH₂)ₚ—CO— or a polyethylene glycol of formula —NH—(O—CH₂—CH₂)ₚ—CO— or a diradical comprising from one to five amino acids, wherein p is an integer comprised between 1 and 20.

4. The compound of formula (I) according to claim 1, wherein R² is —SO₃H.

5. The compound of formula (I) according to claim 1, wherein R² is a group —CONH—Y, wherein Y is selected from the group consisting of

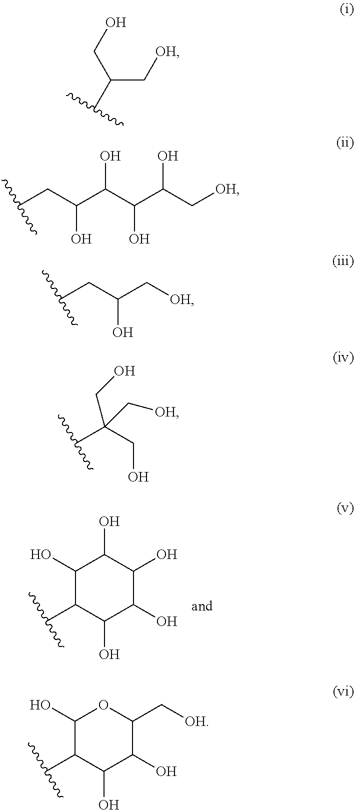

6. The compound of formula (I) according to claim 5 wherein Y is a group

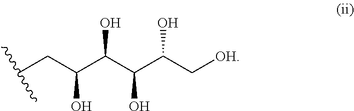 [SMILES: *C[C@H](O)[C@@H](O)[C@H](O)[C@H](O)CO]

7. The compound of formula (I) according to claim 1, wherein both R¹ are a group —(CH₂)₄—SO₃H.

8. The compound of formula (I) according to claim 1, which is represented by the formula (Ia) 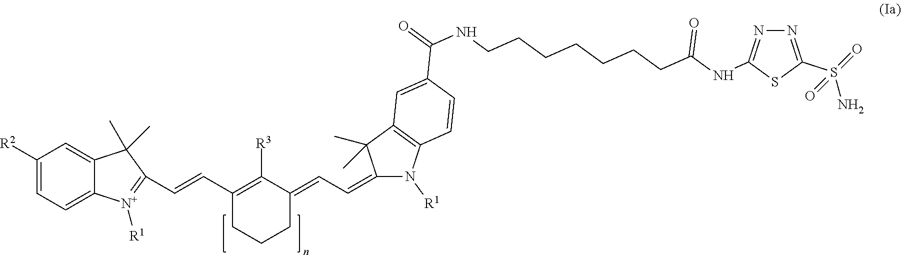
wherein R¹, R², R³ and n are as defined in claim 1.

9. The compound of formula (I) according to claim 1, which is represented by the formula (Ib) 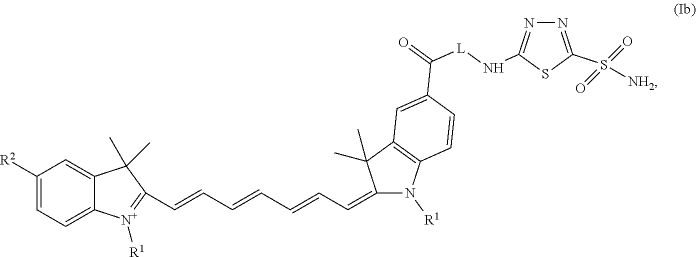
wherein R¹, R² and L are as defined in claim 1.

10. The compound of formula (I) according to claim 1, which is represented by the formula (Ic) 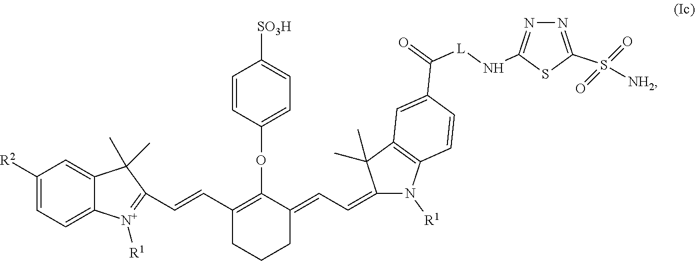
wherein R¹, R² and L are as defined in claim 1.

11. A method for the detection and demarcation of tumor tissue during diagnostic, interventional imaging and intraoperative procedures in a subject in need thereof comprising administering to the subject a compound of formula (I) according to claim 1, irradiating a body of the subject with a wavelength, and visualizing said compound.

12. The method according to claim 11 wherein the detection and demarcation of the tumor tissue is carried out under near-infrared (NIR) radiation.

13. The method according to claim 11, wherein said tumor is a tumor selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer.

14. A pharmaceutical diagnostic composition comprising a compound of formula (I) as defined in claim 1 and at least one pharmaceutically acceptable carrier or excipient.

15. A diagnostic kit comprising at least one compound of formula (I) as defined in claim 1 together with additional adjuvants thereof for implementing a biomedical optical imaging application.

16. A diagnostic kit comprising a pharmaceutical composition as defined in claim 14 together with additional adjuvants thereof for implementing a biomedical optical imaging application.
